(19) United States
(12) Patent Application Publication
(10) Pub. No.: US 2008/0008999 A1
(43) Pub. Date: Jan. 10, 2008

(54) Process for detecting the existence of mesenchymal chrondrosarcoma

(76) Inventor: William D. Hankins, Monrovia, MD (US)

Correspondence Address: HOWARD J. GREENWALD P.C., 349 W. COMMERCIAL STREET SUITE 3075, EAST ROCHESTER, NY, 14445-2408, US

(21) Appl. No.: 11/594,265

(22) Filed: Nov. 8, 2006

Related U.S. Application Data
(63) Continuation-in-part of application No. 11/108,310, filed on Apr. 18, 2005, now abandoned.
(60) Provisional application No. 60/563,326, filed on Apr. 19, 2004.

(51) Int. Cl. C12Q 1/68; C12Q 1/02; G01N 33/53

(52) U.S. Cl. 435/6; 435/29; 435/7.1

(57) ABSTRACT

A process for detecting Mesenchymal Chondrosarcoma in a biological organism, comprising detecting, in a sample that contains Mesenchymal Chondrosarcoma cells obtained from a subject a first product indicative of elevated expression of a fibroblast growth factor receptor gene or a second product indicative of elevated amounts of a fibroblast growth factor receptor (FGFR-L1), wherein detection of said first or second product in elevated expression or amount, respectively, compared to a control sample containing normal or benign Mesenchymal Chondrosarcoma cells indicates the presence of Mesenchymal Chondrosarcoma in said subject. The sample is preferably obtained by a process comprising the steps of: (a) obtaining a tissue sample from a living biological organism, (b) disaggregating said tissue sample to produce disaggregated fragments of tissue sample whose maximum dimension is less than about 5 millimeters, wherein said tissue sample is disaggregated within about 10 minutes of the time said tissue sample is obtained from said biological organism, and (c) disposing said disaggregated tissue fragments in a sterile environment within a container, wherein said sterile environment is comprised of oxygen and a solution comprised of at least one cell type specific viability factor.

### CROSS-REFERENCE TO RELATED PATENT APPLICATIONS

This patent application is continuation-in-part of applicant's copending patent application 11/108,310, filed on Apr. 18, 2005 that, in turn, claimed priority based upon provisional patent application 60/563,326 (filed on Apr. 19, 2004). The disclosure of these prior patent applications is hereby incorporated by reference into this specification.

### FIELD OF THE INVENTION

A process for detecting the existence of Mesenchymal Chrondrosarcoma comprising the steps of analyzing tumor cells and determining the extent to which such tumor cells contain fibroblast growth factor receptor-like 1 protein. When such protein is expressed at least 1,000 percent more than in non-cancerous cells, such overexpression is an indicium of the existence of the Mesenchymal Chrondrosarcoma cancer.

### BACKGROUND OF THE INVENTION

Sarcomas are malignant tumors of mesenchymal origin; there are about 15,000 newly diagnosed soft tissue and bone sarcomas new diagnosed in the United States every year. See, e.g., an article by Kristin Baird et al, “Gene Expression Profiling of Human Sarcomas: Insights into Sarcoma Biology,” Cancer Research 2005; 65:(20), published Oct. 15, 2005.

Mesenchymal Chorondrosacrcoma has been discussed in the patent literature. Reference may be had, e.g., to published United States patent applications 20020009767A1 (Frozen tissue microarrays and methods for using the same), 20020168639A1 (Profile array substrates), and 20030049701 A1 (Oncology tissue micoarrays). The entire disclosure of each of these published United States patent applications is hereby incorporated by reference into this specification.

To the best of applicants knowledge, the prior art has not provided a relatively simple analytical technique for determining the presence of Mesenchymal Chrondrosarcoma. It is an object of this invention to provide such a technique.

### SUMMARY OF THE INVENTION

In accordance with one embodiment of this invention, there is provided a process for detecting Mesenchymal Chondrosarcoma in a biological organism, comprising detecting, in a sample that contains Mesenchymal Chondrosarcoma cells obtained from a subject a first product indicative of elevated expression of a fibroblast growth factor receptor gene or a second product indicative of elevated amounts of a fibroblast growth factor receptor (FGFR-L1), wherein detection of said first or second product in elevated expression or amount, respectively, compared to a control sample containing normal or benign Mesenchymal Chondrosarcoma cells indicates the presence of Mesenchymal Chondrosarcoma in said subject. The sample is preferably obtained by a process comprising the steps of: (a) obtaining a tissue sample from a living biological organism, (b) disaggregating said tissue sample to produce disaggregated fragments of tissue sample whose maximum dimension is less than about 5 millimeters, wherein said tissue sample is disaggregated within about 10 minutes of the time said tissue sample is obtained from said biological organism, and (c) disposing said disaggregated tissue fragments in a sterile environment within a container, wherein said sterile environment is comprised of oxygen and a solution comprised of at least one cell type specific viability factor.

### BRIEF DESCRIPTION OF THE DRAWINGS

The invention will described by reference to the following drawings, in which like numerals refer to like elements, and in which:

FIG. 1 is a flowchart illustrating one preferred process of the invention;

FIG. 2 is a flowchart of another preferred process of the invention;

FIG. 3 is a map of a coordinate system in which the lineage of a particular cell is traced; Hankins to improve Sun am FIG. 4 is a schematic representation of a single ray on the coordinate system of FIG. 2;

FIG. 5 is a flow diagram of a preferred process for tissue preservation, expansion and physiological analyses in which harvested tissue is utilized;

FIG. 6 is a schematic of one preferred apparatus of the invention;

FIG. 7 is a schematic illustration of device utilized in the measurement of the optical properties of cells;

FIG. 8 is a representation of graphs illustrative of the information derived from the optical properties of cells;

FIG. 9 is a representation of graphs illustrative of the information derived from the optical properties of cells;

FIG. 10 is a representation of graphs illustrative of the information derived from the optical properties of cells;

FIG. 11 is a representation of graphs illustrative of the information derived from the optical properties of cells;

FIG. 12 is a representation of graphs illustrative of the information derived from the optical properties of cells;

FIG. 13 is a representation of graphs illustrative of the information derived from the optical properties of cells;

FIG. 14 is a flow diagram of another preferred process of the invention;

FIG. 15 is a schematic of a preferred device for measuring the transmittance of a cell culture;

FIG. 16 is a schematic of another device for measuring the optical properties of a cell culture;

FIG. 17 is a schematic of yet another device for measuring the optical properties of a cell culture;

FIG. 18 is an enlarged view of a portion of the device of FIG. 17;

FIG. 19 is an illustrative graph of one preferred process;

FIG. 20 is a schematic of a preferred embodiment of the process; and

FIG. 21 is a schematic of a preferred embodiment of the process.

### DESCRIPTION OF THE PREFERRED EMBODIMENTS

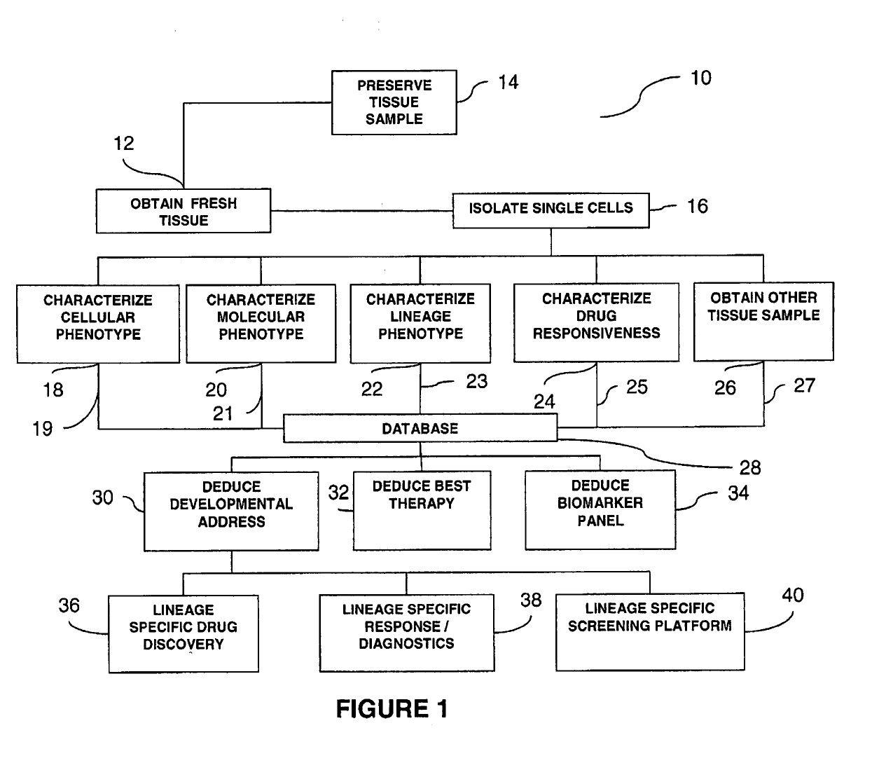

FIG. 1 is a flow diagram of one preferred process 10 of the invention. Referring to FIG. 1, and to the preferred embodiment depicted therein, in step 12 of such process, fresh tissue is obtained from a viable biological organism such, as, e.g., a human being. The tissue may be, e.g., tissue from a heart, lung, blood, liver, brain, hair, etc. The tissue may be normal tissue and/or abnormal tissue. As the term is used in this specification, the term tissue refers to an aggregate of cells and intercellular material that forms a definite structure in which the cells are generally of similar structure and function.

In one embodiment, the tissue is tissue from a malignant tumor. As is known to those skilled in the art, the term malignant is descriptive of tumor that metastasizes and endangers the life of an organism.

In another embodiment, the tissue is tissue that is not malignant but is otherwise abnormal. Thus, the tissue may be tissue infected with a virus or bacteria, or tissue that is malfunctioning (such as in, e.g., hypothyroidism), etc.

In yet another embodiment, the tissue is tissue that is neither malignant nor abnormal but is normal in every respect.

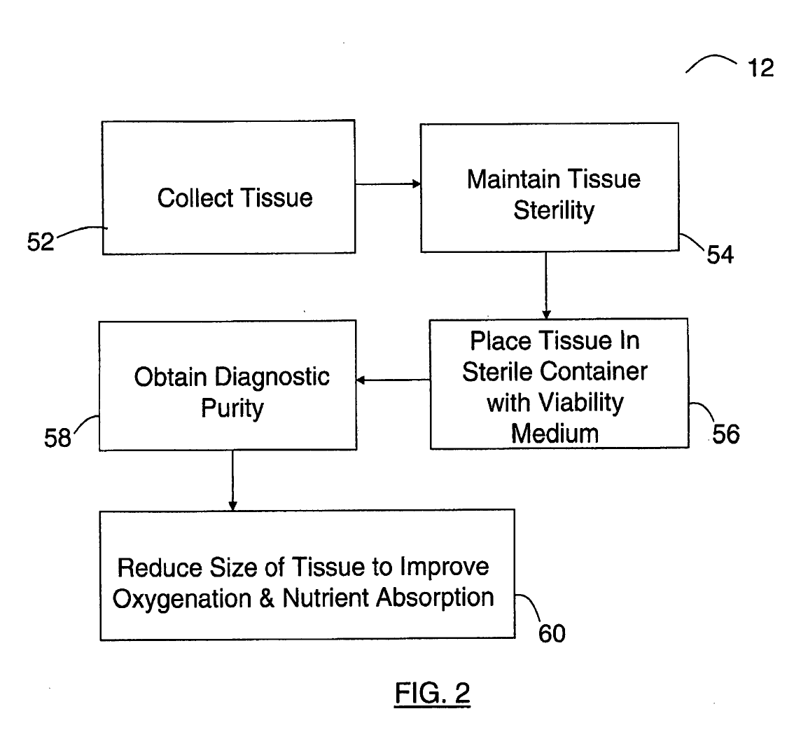

Referring again to step 12 in FIG. 1, in another embodiment, the tissue is obtained from one or more microorganisms such as, e.g., a bacterium, a fungus, a virus, etc. This step 12 is shown in greater detail in FIG. 2.

Referring to FIG. 2, and to the preferred embodiment depicted therein, one may collect the desired tissue by conventional means as shown in step 52. Thus, e.g., one may use one or more of the tissue collection methods disclosed in U.S. Pat. No. 5,624,418 (collection and separation device), U.S. Pat. No. 6,139,508 (articulated medical device), U.S. Pat. No. 6,036,698 (expanded ring percutaneous tissue removal device), U.S. Pat. No. 6,689,145 (apparatus for collecting and staging tissue), U.S. Pat. No. 6,702,831 (excisional biopsy devices and methods), U.S. Pat. No. 6,468,226 (remote tissue biopsy apparatus and associated methods), U.S. Pat. No. 6,022,362 (excisional biopsy devices and methods), U.S. Pat. No. 6,440,147 (excisional biopsy devices and methods), U.S. Pat. No. 5,782,764 (fiber composite invasive medical instruments), U.S. Pat. No. 4,966,162 (flexible endoscope assembly), U.S. Pat. No. 5,449,001 (biopsy needle), U.S. Pat. No. 6,471,709 (expandable ring percutaneous tissue removal device), U.S. Pat. No. 5,338,294 (urological evacuator), U.S. Pat. No. 5,290,303 (surgical cutting instrument), U.S. Pat. No. 5,275,609 (surgical cutting instrument), U.S. Pat. No. 5,183,052 (automatic biopsy instrument with cutting cannula), U.S. Pat. No. 5,569,284 (morecellator), U.S. Pat. No. 5,409,454 (apparatus for atherectomy), and the like. The entire disclosure of each of these United States patents is hereby incorporated by reference into this specification.

Referring to step 54 in FIG. 2, the desired tissue is collected, preferably in a sterile manner, and the sterility of the tissue so collected is maintained. As those who are knowledgeable in the art are aware, the term sterile means free from living germs or microorganisms. Thus, by way of illustration and not limitation, conventional sterile operating room procedures may often be used to insure sterile collection of the tissue from the patient's body. Reference may be had, e.g., to U.S. Pat. No. 6,322,533, “Apparatus for two-path distribution of a sterile operating fluid . . . .” The entire disclosure of this United States patent is hereby incorporated by reference into this specification.

In one preferred embodiment, and referring to step 56 of FIG. 2, after the desired tissue has been removed from the biological organism, it is placed in a sterile container along with a viability medium. The sterile container can be a conventional container, such as a test tube or a Petri dish and the like, that has undergone sterilization. Reference may be had, e.g., to U.S. Pat. No. 3,698,450 (sterile container filling mechanism), U.S. Pat. No. 3,715,047 (silicone stopper for sterile container), U.S. Pat. No. 3,941,245 (sterile container for enclosing a contaminated article), U.S. Pat. No. 3,988,873 (method for enclosing a contaminated article in a sterile container), U.S. Pat. No. 4,056,129 (closable sterile container), U.S. Pat. No. 4,124,141 (sterile container), U.S. Pat. No. 4,982,615 (sterile container for collecting biological samples for purposes of analysis), U.S. Pat. No. 5,178,278 (sterile container with tear-away throat), U.S. Pat. No. 5,462,526 (flexible sterile container), U.S. Pat. No. 5,492,243 (sterile container), U.S. Pat. No. 6,371,326 (sterile container for medical purposes), and the like. The entire disclosure of each of these United States patents is hereby incorporated by reference into this specification.

Referring again to FIG. 2, as those that are knowledgeable in the art are aware, sterilization is the complete destruction of all bacteria and other infectious organisms in an industrial, food, or medical product; it must be followed by aseptic packaging to prevent recontamination, usually by hermetic sealing. The sterilization can be accomplished through conventional methods involving either wet or dry heat, the use of chemicals such as formaldehyde and ethylene oxide filtration, and irradiation by UV or gamma radiation.

In one preferred embodiment, the desired tissue is placed in the sterile container within 3 hours of removal from the biological organism. In another preferred embodiment, the desired tissue is placed in the sterile container within about 1 hour of removal from the biological organism. In another preferred embodiment, the desired tissue is placed in the sterile container within about 15 minutes of removal from the biological organism.

Referring again to step 56 of FIG. 2, and in the preferred embodiment depicted therein, the desired tissue is placed in an enhanced viability medium which is comprised of a viability factor that, preferably, is essential for the cell's viability. As such term is used in this specification, the term “viability factor” refers to a factor that is required for the cell's viability and whose absence will lead to the cell's death. Reference may be had, e.g., to articles by O.S. Frankfurt et al. (“Protection from Apoptotic Cell Death by Inerleukin-4 is Increased in Lymphocytic Leukemia Patients,” Leuk. Res. 21:9-16, Elsevier Science, Ltd., January, 1997), by A. Horigome et al. (“Tacrolimus-inducted apoptosis and its prevention by interleukin blood mononuclear cells,” Immunopharmacology, 39:21-30, Elsevier Science B.V.), by H. Lindner et al. (“Peripheral Blood Mononuclear Cells Induce Programmed Cell Death . . . ,” Blood 89:1931-1938.

The viability factor may be a viability hormone such as, e.g., a stem cell viability factor. Reference may be had, e.g., to U.S. Pat. No. 5,601,056 (use of stem cell factor interleukin-6 . . . to induce the development of hematopoietic stem cells), U.S. Pat. No. 5,786,323 (use of stem cell factor and soluble interleukin-6 receptor to induce the development of hematopoietic stem cells), U.S. Pat. No. 5,861,315 (use of stem cell factor and soluble interleukin-6 receptor for the ex vivo expansion of hematopoietic multipotential cells), U.S. Pat. No. 5,885,962 (stem cell factor analog compositions), U.S. Pat. No. 6,824,973 (method of promoting stem cell proliferation or survival by contacting a cell with a stem cell factor-like polypeptide), U.S. Pat. No. 6,852,313 (method of stimulating growth of melanocyte cells by administering stem cell factor), and the like. The entire disclosure of each of these United States patents is hereby incorporated by reference into this specification.

By way of further illustration, the viability hormone may be erythropoietin. As is known to those skilled in the art, erythoropoietin is a glycoprotein mitogen and hormone with a molecular weight of about 23,000 Daltons that is produced by the kidneys and that stimulates the formation of erythrocytes; and its presence is essential for the viability of erythroid cells. Reference may be had, e.g., to U.S. Pat. No. 5,830,851 (methods of administering peptides that bind to the erythorpoietini receptor), U.S. Pat. No. 5,986,047 (peptides that bind to the erythropoietin receptor), U.S. Pat. No. 6,531,121 (protection and enhancement of erythropoietin-responsive cells, tissues, and organs), and the like. The entire disclosure of each of these United States patents is hereby incorporated by reference into this specification.

By way of further illustration, the viability hormone may be a follicle stimulating hormone. As is known to those skilled in the art, follicle stimulating hormone is the gonadotropic protein hormone, secreted by the anterior lobe of the pituitary gland, that stimulates the growth of ovarian follicles and the secretion of estadiol in the female and spermatogenesis in the male; its presence is essential for the viability of ovarian follicular cells. Reference may be had, e.g., to U.S. Pat. No. 5,744,448 (human follicle(human follicle stimulating hormone receptor), U.S. Pat. No. 5,767,067 (follicle stimulating hormone), U.S. Pat. No. 6,306,654 (follicle stimulating hormone-glyosylation analogs), U.S. Pat. No. 6,737,515 (follicle stimulating hormone-glycosylation analogs), and the like The entire disclosure of each of these United States patents is hereby incorporated by reference into this specification.

By way of yet further illustration, the viability hormone may be a melanocyte stimulating hormone. As is known to those skilled in the art, such a hormone is one of two peptide hormones, denoted alpha and beta, that are produced by the posterior lobe of the pituitary gland and that have a darkening effect by causing the dispersion of melanin pigments in the melanocytes. Reference may be had, e.g., to U.S. Pat. No. 5,126,327 (melanocyte-stimulating hormone inhibitor), U.S. Pat. No. 5,849,871 (alpha-melanocyte stimulating hormone receptor), U.S. Pat. No. 6,268,221 (melanocyte stimulating hormone receptor), U.S. Pat. No. 6,660,856 (antagonists of alpha-melanocyte stimulating hormone and methods based thereon), and the like. The entire disclosure of each of these United States patents is hereby incorporated by reference into this specification.

By way of yet further illustration, the viability hormone may be thyrotropin. As is known to those skilled in the art, thyrotropin is a protein hormone, secreted by the anterior lobe of the pituitary gland, that stimulates the synthesis of thyroid hormones and the release of thyroxine by the thyroid gland; and its presence is essential for the viability of thyroid epithelial cells Reference may be had, e.g., U.S. Pat. No. 3,959,248 (analogs of thyrotropin-releasing hormone), U.S. Pat. No. 4,493,828 (use of thyrotropin releasing hormone and related peptides as poultry growth promotants), U.S. Pat. No. 5,864,420 (thyrotropin-releasing hormone analogs), U.S. Pat. No. 5,879,896 (method of screening for inhibitors of human thyrotropin releasing hormone receptor), U.S. Pat. No. 6,441,133 (thyrotropin-releasing hormone receptor), and the like. The entire disclosure of each of these United States patents is hereby incorporated by reference into this specification.

By way of yet further illustration, the viability hormone may be epidermal growth factor. As is known to those skilled in the art, epidermal growth factor is a polypeptide mitogen, with a molecular weight of about 6400, that stimulates the proliferation of epidermal and epithelial tissues and the presence of which is required for the viability of such tissues. Reference may be had, e.g., to U.S. Pat. No. 5,960,820 (epidermal growth factor receptor targeted molecules), U.S. Pat. No. 6,129,915 (epidermal growth factor receptor antibodies), U.S. Pat. No. 6,255,452 (epidermal growth factor inhibitor), and the like. The entire disclosure of each of these United States patents is hereby incorporated by reference into this specification.

As will be apparent, one can determine by conventional means whether a particular factor, such as, e.g., a prospective viability hormone, is indeed essential for the survival of a particular cell by testing the viability of such cell in both the presence of and the absence of such factor. Reference may be had, e.g., to U.S. Pat. No. 6,843,980, that describes “Methods for using annexin for detecting cell death in vivo and treating associated conditions.” Reference also may be had to U.S. Pat. No. 5,185,450 (tetrazolium compounds for cell viability assays), U.S. Pat. No. 5,314,805 (dual-fluorescence cell viability assay using ethidium homodimeer and calcein AM), U.S. Pat. No. 6,403,378 (cell viability assay reagent), and the like. The entire disclosure of each of these United States patents is hereby incorporated by reference into this specification.

The disclosure contained in U.S. Pat. No. 6,403,378 is of interest. As is disclosed in this patent, “Two dyes are generally used to stain cells in a suspension for viability analysis. One dye consists of a membrane permeant DNA dye that labels all intact cells in a suspension, whereby they emit light at one wavelength. A non-permeant DNA dye labels all dead cells.”

U.S. Pat. No. 6,403,378 also discloses that “In one method of analysis, the cells in the cell suspensions are stained and a traditional hemacytometer is used to differentiate the cells. Another analysis system utilizes dual-color fluorescence in combination with forward light scatter to determine the concentration of nucleated cells and cell viability. Cells are analyzed by providing relative movement between the sample suspension containing the cells and an excitation light beam, whereby labeled cells pass through the light beam and emit light at a wavelength characteristic of the permeant and non-permeant dye. The detection system includes filters and detectors which detect the light emitted at the two wavelengths. The cells also scatter light, whereby all particles in the sample suspension are detected. Once a cell has been detected on the permeant dye channel, the light scatter profile is evaluated to assure that the cell is of sufficient size to be an intact cell and not simply a free nucleus or other cell fragment. The second dye permeates all cells with damaged or “leaky” membranes. The dye emits fluorescent light at a different wavelength range than that of the cells stained with permeant dye. In this manner all cells are detected by detecting the light emitted by the second dye at one wavelength, and non-viable cells are detected by detecting light emitted by the permeant dye at the other wavelength. Thus, an absolute count of cells and percent viability can be obtained from the data.”

U.S. Pat. No. 6,403,378 also discloses that “To obtain reliable results for different cell concentrations, using a two-dye method it is necessary to carefully control the amount of each of the dyes used to stain or tag the cells. This is a time-consuming procedure and may lead to variability in results obtained.”

As will be apparent to those skilled in the art, when a particular hormone is found to be essential for the viability of a particular cell, it is deemed to be a cell type specific viability hormone.

Referring again to FIG. 2, and in step 56 thereof, one may use other known means for insuring viability. Various media for maintaining viability are well known to those skilled in the art. Reference may be had, e.g., to U.S. Pat. No. 5,543,316, for an “Injectable culture medium for maintaining viability of myoblast cells.” The entire disclosure of such United States patent is hereby incorporated by reference into this specification.

In one embodiment, the base viability medium is be a sterile saline solution, or a balanced salt solution, or a glucose containing culture medium, serum, or the like.

Referring again to FIG. 2, and in step 56 thereof, the cell type specific hormone preferably is present in the viability medium at a concentration from about 0.01 to about 10 micrograms per milliliter; in one aspect of this embodiment, the hormone is present in a range of from about 0.1 to about 5 micrograms per milliliter. In yet another embodiment, the hormone is present at a concentration of from about 0.3 to about 3 micrograms per milliliter.

In one preferred embodiment, the desired tissue is maintained with at least about 90% viability. In another preferred embodiment, the desired tissue is maintained with at least about 95% viability. In another preferred embodiment, the desired tissue is maintained with at least about 99% viability. The desired tissue is preferably tested for viability using the tryphan blue exclusion test as is described in U.S. Pat. No. 5,739,274 (active component of parathyroid hypertensive factor), U.S. Pat. No. 6,008,007 (radiation resistance assay for predicting treatment response and clinical outcome), U.S. Pat. No. 6,261,795 (radiation resistance assay for predicting treatment response and clinical outcome), U.S. Pat. No. 6,447,810 (composition of multipurpose high functional alkaline solution composition, preparation thereof, and for the use of nonspecific immunostimulator), U.S. Pat. No. 6,673,375 (composition of multipurpose high functional alkaline solution composition, preparation thereof, and for the use of nonspecific immunostimulator), and U.S. Pat. No. 6,699,851 (cytotoxic compounds and their use). The entire disclosure of these United States patents are hereby incorporated by reference.

Referring to step 58 in FIG. 2, in one preferred embodiment, the desired tissue is processed to obtain a diagnostic purity. As is known by those skilled in the art, diagnostic purity refers to characterizing the cells that are purified from the surgical tissue (containing the tumor and some normal tissue) and at least 90 percent of the cells are the same as the original diagnosis. As is known to those skilled in the art, one may determine purity by visual observation of morphology under a microscope. In one preferred embodiment, the desired cells are tumor cells and not the surrounding normal cells. In one preferred embodiment, a diagnostic purity of at least about 90 percent is obtained. In another preferred embodiment, a diagnostic purity of at least about 95 percent is obtained. In another preferred embodiment, a diagnostic purity of at least about 99 percent is obtained. This diagnostic purity is preferably obtained by separating the desired tissue from the surrounding tissue. In one preferred embodiment, the desired tissue is a tumor and the surrounding tissue is normal.

Referring again to step 58 in FIG. 2, in one preferred embodiment, the purity of the desired tissue can be measured by conventional means such as one or more of the processes described in U.S. Pat. No. 5,741,648 (cell analysis method using quantitative fluorescence image analysis) and U.S. Pat. No. 5,733,721 (cell analysis method using quantitative fluorescence image analysis); the entire disclosure of each of these United States patents is hereby incorporated by reference into this specification.

U.S. Pat. No. 5,733,721 describes a quantitative fluorescence image analysis (QFIA) method and claims (in claim 1): “A method of analyzing a cell sample derived from urine or from a bladder wash, comprising: providing a prepared slide, the prepared slide having been prepared by applying a portion of a cell sample to a slide, the portion of the cell sample treated with a fixative composition comprising a salt of ethylenediaminetetraacetic acid effective in inhibiting formation of substantially all of the crystals in the cell sample prior to application of the portion of the cell sample to the slide leaving the prepared slide substantially free of crystals for improving microscopic analysis of the cell on the prepared slide, then treating the slide with a fluorescent label for labeling the cytological marker to form a labeled cytological marker; irradiating a portion of the prepared slide with an amount of an excitation wavelength of light effective in causing the fluorescent label in a cell to emit fluorescent light having an emission wavelength for forming a field image; using a microscope means to select cell images on the field image; obtaining a number related to the selected cell images; and outputting the number for use in classifying the cell sample.” U.S. Pat. No. 5,733,721 further claims (in claim 23): “A method of analyzing a cell sample derived from urine or from a bladder wash, comprising: providing a prepared slide, the prepared slide having been prepared by applying a portion of a cell sample to a slide, the portion of the cell sample treated with a fixative composition comprising a salt of ethylenediaminetetraacetic acid effective in inhibiting formation of substantially all of the crystals in the cell sample prior to application of the portion of the cell sample to the slide leaving the prepared slide substantially free of crystals for improving microscopic analysis of the cell on the prepared slide, then treating the slide with a first fluorescent label for labeling the first cytological marker to form a labeled first cytological marker and the second fluorescent label for labeling the second cytological marker to form a labeled second cytological marker; irradiating a first portion of the prepared slide with an amount of a first excitation wavelength of light effective in causing the first fluorescent label in the cell to emit fluorescent light having a first emission wavelength for forming a first field image; using a microscope means to select first cell images on the first field image; obtaining a first number related to the selected first cell images; irradiating the second portion of the prepared slide with a second excitation wavelength of light effective in causing the second fluorescent label to emit fluorescent light having a second emission wavelength for forming a second field image wherein the second portion may be the same as the first portion; using the microscope means to select second cell images on the second field image; obtaining a second number related to the selected second cell images; and outputting the first number and the second number for use in classifying the cell sample.”

Referring to step 60 in FIG. 2, in one preferred embodiment, the desired tissue is separated into smaller pieces to allow for oxygenation of the cells of the tissue and to allow for nutrient absorption by the cells of the tissue. In one preferred embodiment, the desired tissue is sliced into thin slices of preferably from about 2 millimeters thickness. In another preferred embodiment, the desired tissue is sliced into thin slices of preferably from about 0.50 millimeters thickness or less. In another preferred embodiment, the desired tissue is sliced into thin slices of from about 0.01 millimeter or less. One may use conventional means to disaggregate the tissue sample. Reference may be had, e.g., to U.S. Pat. No. 3,941,317, for a “Method and apparatus for tissue disaggregation.” The entire disclosure of this United States Patent hereby incorporated by reference into this specification.

Referring again to FIG. 1, after the desired tissue sample is obtained in step 12, then it may either be preserved (in step 14), and/or isolated single cells may be obtained in step 16. One may isolate single cells by conventional means such as, e.g., preparing single cell suspensions. Reference may be had to U.S. Pat. No. 4,350,768 (method for preparing single cell suspension), U.S. Pat. No. 4,413,059 (apparatus for preparing single cell suspension), U.S. Pat. No. 5,728,580 (method for inducing single cell suspension in insect cell lines), U.S. Pat. No. 5,744,363 (method for establishing a tumor cell line by preparing single cell suspension of tumor cells from tumor biopsies), U.S. Pat. No. 6,103,526 (Spodoptera frugiperda single cell suspension cell line in serum-free media), and the like. The entire disclosure of each of these United States patents is hereby incorporated by reference into this specification.

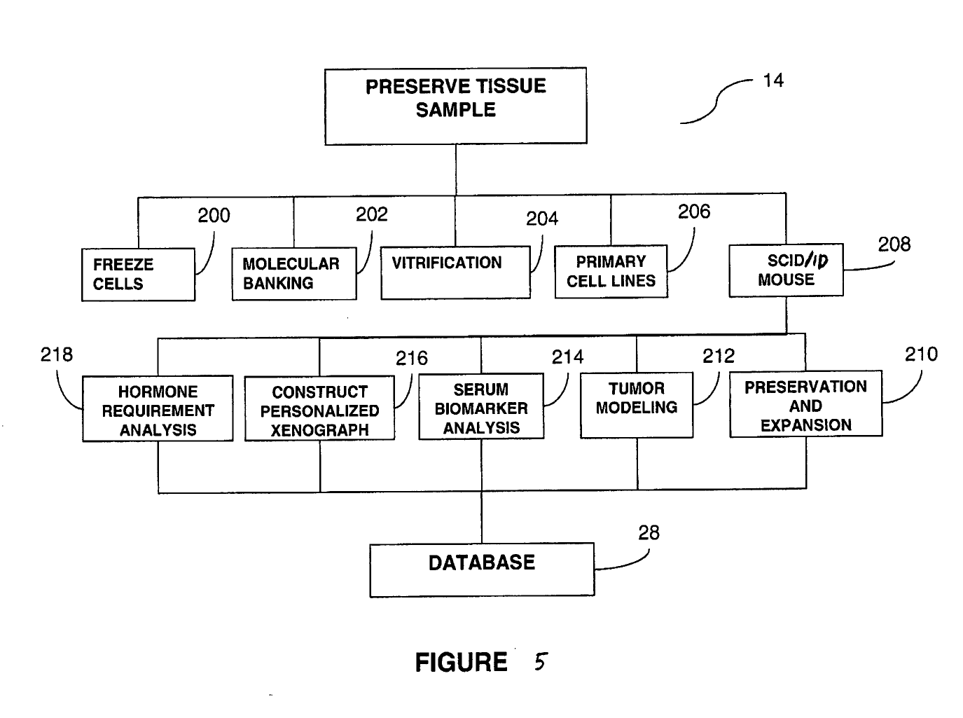

Referring to FIG. 1, and to the preferred embodiment depicted therein, in step 14 of such process, the desired tissue is preserved in a viable state and the tissue viability is preferably tested using the tryphan blue exclusion test. By way of illustration, an example of a method of tissue preservation is claimed in U.S. Pat. No. 6,569,615 (composition and methods for tissue preservation); the entire disclosure of this United States patent is hereby incorporated by reference. One preferred means of preserving such tissue and/or cells will be discussed elsewhere in this specification with reference to FIG. 5.

Referring to step 16 in FIG. 1, and to the preferred embodiment depicted therein, in one preferred embodiment, the cells are obtained by one or more of the processes described in U.S. Pat. No. 5,733,721 (cell analysis method using quantitative fluorescence image analysis), U.S. Pat. No. 5,741,648 (cell analysis method using quantitative fluorescence image analysis), U.S. Pat. No. 5,824,495 (cell fixative and preparation, kit and method) U.S. Pat. No. 6,194,165 (cell fixative and preparation composition, kit and method), and U.S. Pat. No. 6,372,450 (method of treating cells); the entire disclosure of each of these United States patents is hereby incorporated by reference into this specification.

In such step 16, certain single cells are isolated. The single cells may be obtained by conventional means. To this end, one may use one or more of the processes claimed in U.S. Pat. No. 5,827,735 (pluripotent mesenchymal stem cells), U.S. Pat. No. 6,420,105 (method for analyzing molecular expression of function in an intact single cell), U.S. Pat. No. 6,541,247 (method for isolating ependymal neural stem cells), U.S. Pat. No. 6,686,197 (method for producing preparations of mature and immature pancreatic endocrine cells), and the like. The entire disclosure of each of these United States patents is hereby incorporated by reference into this specification.

By way of further illustration, and as is claimed in U.S. Pat. No. 6,077,684, one may conduct the step of “isolating a single cell suspension from the sample.” Thus, e.g., and as is disclosed in columns 14 and 15 of such patent, “Prior to any chemotherapy, a sample of venous blood (e.g., 1-30 ml) or a sample of bone marrow (e.g., 2-20 ml) is obtained by direct needle aspiration under sterile conditions. The samples are drawn into a heparinized syringe and diluted with RPMI-1640 medium that contains no phenol red. The mononuclear fraction of each sample is isolated by centrifugation using Ficoll-Hypaque. If erythrocytes contaminate the mononuclear cell fraction, then they are removed by treatment with red cell lysis buffer. After washing three times in phosphate buffered saline, an aliquot of the mononuclear cells is analyzed by either light microscopy or flow cytometry for purity and viability. The specific MAb's that recognized the leukemia cells in the diagnostic testing are used to check purity while 7-amino-actinomycin D (7AAD) is used to check viability. If purity and viability are both greater than 90%, then the cells are aliquoted for the present assays and for cryopreservation in RPMI-1640 containing 20% fetal bovine serum and 10% dimethylsulfoxide. Greater than 90% purity and viability would be expected in most cases with a high leukemic cell count in either the blood or bone marrow. If the mononuclear cell fraction purity is less than 90%, then the cells are further purified. T-lymphocytes and monocytes are removed by negative selection using immunomagnetic separation. MAb's to CD2 for T-cell removal and CD14 for monocyte removal and Dynabeads (Dynal, Inc.) are used in those cases in which the diagnostic immunophenotyping shows that the leukemic cells lack these surface antigens. After these immunomagnetic separations, the leukemic cell population will again be tested for purity.

In one embodiment, the tissue is preferably rendered into smaller pieces and then digested with a series of enzymes (such as, e.g., trypsin, collagenase, lipase, and the like) to disaggregate the tissue into stromal cell, connective tissue, and tumor cells such that the tumor cells can then be readily isolated.

As the term is used in this specification, the term tissue refers to an aggregate of cells and intercellular material that forms a definite structure in which the cells are generally of similar structure and function.

As is known to those skilled in the art, digestion involves the chemical or enzymatic hydrolysis of macromolecules. Reference may be had, e.g., U.S. Pat. No. 4,350,768 (method for preparing single cell suspensions), U.S. Pat. No. 4,413,059 (apparatus for preparing single cell suspensions), U.S. Pat. No. 5,728,580 (methods and culture media for inducing single cell suspension in insect cell lines), and U.S. Pat. No. 6,420,105 (method for analyzing molecular expression or function in an intact single cell), to descriptions of the enzymatic preparation of single cell suspensions. The patents also refer to mechanical methods of preparing single cell suspensions. The disclosures of these patents are hereby incorporated by reference.

If trypsinization is used, the cells must recover the functionality of the membrane proteins.

In one preferred embodiment, and referring to step 16 of FIG. 1, during the time that the single cells are isolated, it is preferred to expose such cells to an oxygen-containing atmosphere containing at least 1 volume percent of oxygen and, more preferably, at least about 5 volume percent of oxygen. In one embodiment, the cells are maintained in an atmosphere of at least about 10 percent oxygen. In another embodiment, the cells are maintained in an atmosphere of at least about 20 percent oxygen. In one aspect of this embodiment, either oxygen and/or an oxygen-containing gas (such as a mixture of 5 volume percent carbon dioxide and 95 volume percent of oxygen) is bubbled through a cell solution medium comprised of the cells in question to adequately oxygenate substantially all of the cells in the medium.

Referring again to FIG. 1, it is preferred, while conducting the cell isolation step 16, to maintain the cells within a temperature of from about 22 to about 39 degrees Celsius.

It is also preferred, while isolating the single cells in step 16, to continue to contact such cells with the enhanced viability medium introduced in step 12.

In one preferred embodiment, the single cells are isolated from a medium that contains a molecule that tends to prevent apoptosis. As known to those skilled in the art, apoptosis is one of the two mechanisms by which cell death occurs (the other being the pathological process of necrosis). Apoptosis is the mechanism responsible for the physiological deletion of cells, and is characterized by distinctive morphologic changes in the nucleus and cytoplasm, chromatin cleavage at regularly spaced sites, and the endonucleolytic cleavage of genomic DNA at internucleosomal sites. Apoptosis serves as a balance to mitosis in regulating the size of animal tissues and in mediating pathologic processes associated with tumor growth. These molecules that prevent apoptosis are well known and are described, e.g., in an article by H Rui et al., “Activation of the Jak20Stat5 signaling pathway in Nb2 lymphoma cells by an anti-apoptoic agent, aurintricarboxylic acid,” J. Biol. Chem. 1998, Jan. 2; 273 (1):28-32.

Aurintricarboxylic acid is well known and is described, e.g., in U.S. Pat. No. 5,431,185, the entire disclosure of which is hereby incorporated by reference into this specification. As is disclosed in such patent, U.S. Pat. No. 4,007,270 to Bernstein et al. discloses that aurintricarboxylic acid (ATA) and certain of its derivatives and salts are useful as complement inhibitors which play an important role as mediators in immune, allergic, immunochemical and immunopathological reactions. As is well known in the field, the term “complement” refers to a complex group of proteins in body fluids that, working together with antibodies or other factors, play an important role as mediators of immune, allergic, immunochemical and/or immunopathological reactions. The reactions in which complement participates take place in blood serum or in other body fluids, and hence are considered to be humoral reactions. Aurins (free acid and ammonium salt) may be prepared according to the method of G. B. Heisig and W. M. Lauer, Org. Syn. Coll. Vol. 1 (second Ed.), 54-55 (1932); Holaday, D. A., J. Am. Chem. Soc., 62, 989 (1940); The Merck Index, 8th Ed. (1968), page 42; and Caro, Ber., 25, 939 (1892). Esterification with an alcohol and acylation in the presence of an acid provide the derivatives of this invention. The salts of the free acid and acylates may be obtained by treatment thereof with a suitable base in an aqueous alcohol. The patent discloses a method of inhibiting the complement system in blood serum subjecting the serum to aurintricarboxylic acid or its derivatives or salts and that ATA has anti-inflammatory properties. The patent discloses Aurintricarboxylic acid (ATA) is a heterogeneous mixture of polymers that forms when salicylic acid is treated with formaldehyde, sulfuric acid and sodium nitrite (see Cushman, M. et al. “Preparation and Anti-HIV Activities of Aurintricarboxylic Acid Fractions and Analogues: Direct Correlation of Antiviral Potency with Molecular Weight”, J. Med. Chem., Volume 34, (1991) pp. 329-337; Cushman, M. et al. “Synthesis and Anti-HIV Activities of Low Molecular Weight Aurintricarboxylic Acid Fragments and Related Compounds”, J. Med. Chem., Volume 34, (1991) pp. 337-342).

In one embodiment, and referring again to step 16 of FIG. 1, the cells are isolated in step 16 while in the presence of an anti-apoptic agent such as, e.g., aurintricarboxylic acid (ATA) and optionally, other agents that promote cell viability. Aurintricarboxylic acid is known to cause cell death, in appropriate concentrations. Thus, e.g., U.S. Pat. No. 5,434,185 describes in claim 1“1. A method for inhibiting angiogenesis in an animal comprising administering an effective amount to inhibit angiogenesis of aurintricarboxylic acid, its analogues, or salts to said animal.” Claim 3 of this patent describes “3. A method according to claim 1, wherein said effective amount comprises about 10 mg/kg body weight of the host aurintricarboxylic acid.” In one preferred embodiment, not shown, the dosage of aurintricarboxylic acid that is known to cause cell death is from 0.01 micromoles to 0.1 micromoles. Thus the aurintricarboxylic acid needs to be applied in doses not approaching this level.

Regardless of the process of isolation of the single cells used in step 16, it is preferred that the single cells so isolated have a viability of at least about 90 percent and a purity of at least about 90 percent. In one embodiment, the viability and the purity is at least about 95 percent. One may determine the viability of the cell samples by tryphan blue exclusion. One may determine purity by visual observation of morphology under a microscope.

Referring again to FIG. 1, and in the preferred embodiment depicted therein, the single cells isolated in step 16 of the process 10 may be used to characterize the cellular phenotype (in step 18), and/or to characterize the molecular phenotype of the cell (in step 20), and/or to characterize the lineage phenotype of the cell (in step 22), and/or to characterize the drug response of the cell (in step 24). Alternatively, or additionally, one may also obtain patient samples for additional analyses or information.

Referring again to FIG. 1, and to step 18 of process 10, the cellular phenotype of the isolated single cells is characterized. As is known to those skilled in the art, the term phenotype refers to the physical appearance and the observable properties of an organism that are produced by the interaction of the genotype with the environment. The cellular phenotype refers to the physical appearance and the observable properties of a cell that are produced by the expression of specific sets of genes and proteins.

One may characterize the cellular phenotype of the isolated single cells by conventional means. Reference may be had, e.g., to U.S. Pat. No. 6,197,523 (method for the detection, identification, enumeration, and confirmation of circulating cancer and/or hematologic progenitor cells in whole blood), U.S. Pat. No. 5,496,704 (in vitro detection of formed elements in biological samples), U.S. Pat. No. 5,403,714 (method for in vitro detection of formed elements in biological samples), U.S. Pat. No. 4,099,917 (process for preparing a cell suspension from blood for discrimination of white blood cells and platelets from other blood particles), and the like. The entire disclosure of each of these United States patents is hereby incorporated by reference into this specification.

As will be apparent, the characterization of the cellular phenotype of the isolated single cells furnish some gross information about the broad lineage of the isolated single cells, i.e., whether such cells are brain cells, breast cells, lung cells, pancreas cells, etc.

Referring again to FIG. 1, and in step 20 thereof, the molecular phenotype of the isolated single cells is characterized. This will furnish more information regarding the broad lineage of the isolated single cells, i.e., whether the single cells are expressing genes and proteins from brain tissue, breast tissue, lung tissue, etc.

One may characterize the molecular phenotype of the isolated single cell populations by conventional means. Thus, e.g., one may use the characterization processes described in U.S. Pat. No. 6,406,630 (treating cancers associated with overexpression of HER-2/neu), U.S. Pat. No. 6,291,496 (treating cancers associated with overexpression of class I family . . . ), U.S. Pat. No. 6,200,760 (method of screening agents as candidates for drugs or sources of drugs), U.S. Pat. No. 5,876,932 (method for gene expression analysis), U.S. Pat. No. 6,203,987 (methods for using co-regulated genesets to enhance detection and classification of gene expression patterns), U.S. Pat. No. 6,406,921 (protein arrays for high-throughput screening), U.S. Pat. No. 6,355,423 (methods and devices for measuring differential gene expression), U.S. Pat. No. 6,475,809 (protein arrays for high-throughput screening), U.S. Pat. No. 6,537,749 (addressable protein arrays), U.S. Pat. No. 6,548,021 (surface-bound, double-stranded DNA protein arrays), U.S. Pat. No. 6,635,423 (informative nucleic acid arrays), U.S. Pat. No. 6,618,679 (methods for analysis of gene expression), U.S. Pat. No. 6,653,135 (dynamic protein signature assay), U.S. Pat. No. 6,696,620 (immunoglobulin binding protein arrays), and the like. The entire disclosure of each of these United States patents is hereby incorporated by reference into this specification.

In one embodiment, and referring again to step 20 of FIG. 1, the molecular phenotype is characterized by the process described in U.S. Pat. No. 6,221,600 (combinatorial oligonucleotide PCR: a method for rapid, global expression analysis), the entire disclosure of which is hereby incorporated by reference into this specification. This patent claims: “A method comprising: a) obtaining a DNA comprising an anchorable moiety; b) cleaving said DNA with a first restriction endonuclease; c) ligating a linker molecule to cleaved DNA produced in step b;d) immobilizing linker ligated DNA through said anchorable moiety; e) cleaving DNA immobilized in step d with a second restriction endonuclease; f) ligating a second linker molecule to DNA cleaved in step e; g) amplifying DNA ligated in step f.” As is disclosed in the abstract of this patent, “The present invention relates to a method for the detection of gene expression and analysis of both known and unknown genes. The invention is a highly sensitive, rapid and cost-effective means of monitoring gene expression, as well as for the analysis and quantitation of changes in gene expression for a defined set of genes and in response to a wide variety of events. It is an important feature of the present invention that no single molecular species of cDNA gives rise to more than one fragment in the collection of products which are subsequently amplified and representative of each expressed gene. This achievement is facilitated by immobilizing the cDNA prior to digesting and then digesting with sequentially with two frequently cutting enzymes. Linker oligomers are ligated to each cut site following the respective digestion. Primers, complementary to the oligomer sequence with an additional 3′ variable sequence are used to amplify the fragments. Using an array of fragments theoretically facilitates the amplification of all of the possible messages in a given sample.”

Referring again to FIG. 1, and in the preferred embodiment depicted therein, in step 22 of process 10 the lineage phenotype of the isolated single cells is characterized. As is known to those skilled in the art, the lineage of a cell is its developmental pathway. Development, as used in this specification, refers to the series of orderly changes by which a mature cell, tissue, organ, organ system, or organism comes into existence. Each cell is part of a developmental pathway that, through a process of differentiation, proliferation, and maturation, produces functional cells from non-functional stem or seed cells.

Thus, e.g., and as is disclosed in U.S. Pat. No. 6,248,587 (the entire disclosure of which is hereby incorporated by reference into this specification), “Mesenchymal stem cells (MSC) are pluripotent progenitor cells that possess the ability to differentiate into a variety of mesenchymal tissue, including bone, cartilage, tendon, muscle, marrow stroma, fat and dermis as demonstrated in a number of organisms, including humans (Bruder, et al., J. Cellul. Biochem. 56:283-294 (1994). The formation of mesenchymal tissues is known as the mesengenic process, which continues throughout life, but proceeds much more slowly in the adult than in the embryo (Caplan, Clinics in Plastic Surgery 21:429-435 (1994). The mesengenic process in the adult is a repair process but involves the same cellular events that occur during embryonic development (Reviewed in Caplan, 1994, supra). During repair processes, chemoattraction brings MSC to the site of repair where they proliferate into a mass of cells that spans the break. These cells then undergo commitment and enter into a specific lineage pathway (differentiation), where they remain capable of proliferating. Eventually, the cells in the different pathways terminally differentiate (and are no longer able to proliferate) and combine to form the appropriate skeletal tissue, in a process controlled by the local concentration of tissue-specific cytokines and growth factors (Caplan, 1994, supra).”

Referring again to FIG. 1, in step 22 the lineage pathway of the isolated single cells is determined. This can be accomplished by conventional means such as, e.g., the processes disclosed in U.S. Pat. No. 5,817,773 (stimulation, production, culturing and transplantation of stem cells by fibroblast growth factors), U.S. Pat. No. 6,248,547 (process for promoting lineage-specific cell proliferation and differentiation), U.S. Pat. No. 6,268,212 (tissue specific transgene expression), U.S. Pat. No. 6,280,724 (composition and method for preserving progenitor cells), U.S. Pat. No. 6,380,458 (cell-lineage specific expression in transgenic zebrafish), U.S. Pat. No. 6,391,297 (differentiation of adipose stromal cells in osteoblasts), U.S. Pat. No. 6,548,299 (lymphoid-tissue specific cell production from hematopoietic progenitor cells in three-dimensional devices), and the like. The entire disclosure of each of these United States patents is hereby incorporated by reference into this specification.

In one preferred embodiment, the lineages of the isolated single cells are analyzed to determine, e.g., the presence of known proteins and/or antigens associated with specific lineages of such cells. By way of illustration, these may include hormone receptors, lineage specific kinases, lineage specific transcription factors and/or regulators, lineage specific gene rearrangements, and the like.

Referring again to FIG. 1, in step 24 of process 10, the drug response of the isolated single cells is characterized. One may use conventional means for determining the drug response of such cells such as, e.g., the means disclosed in U.S. Pat. No. 4,816,395 (method for predicting chemosensitivity of anti-cancer drugs), U.S. Pat. No. 4,937,182 (method for predicting chemosensitivity of anti-cancer drugs), U.S. Pat. No. 6,468,547 (enhancement of tumor cell chemosensitivity), U.S. Pat. No. 6,521,407 (methods for determining chemosensitivity of cancer cells based on expression of negative and positive signal transduction factors), U.S. Pat. No. 6,620,403 (in vivo chemosensitivity screen for human tumors), and the like. The entire disclosure of each of these United States patents is hereby incorporated by reference into this specification.

In one embodiment, the process of U.S. Pat. No. 6,258,553 of Kravtsov is used to effectuate step 24. Claim 1 of this patent describes: “A method of determining the apoptosis-inducing activity of a substance, which comprises: a) measuring the optical density of a first cell culture at more than one time point, wherein the first cell culture was contacted with the substance; b) measuring the optical density of a second cell culture at more than one time point, wherein the second cell culture was not contacted with the substance; and c) determining a net slope, which is the difference between the optical density change over time of the first cell culture and the optical density change over time of the second cell culture; wherein a positive net slope indicates apoptosis-inducing activity of the substance.” The entire disclosure of this United States patent is hereby incorporated by reference into this specification.

Several patents and patent publications have been issued or published in the name of Vladimir D. Kravtsov. These include U.S. Pat. Nos. 6,077,684 and 6,258,553, International publications numbers WO 02/40702A2, WO 02/42749 A2, WO 02/46750 A2, WO 02/46751 A2, and Australian patent publications AU0225874A5, AU0239491A5, and AU0239771A5. The entire disclosure of each of these patents and patent applications is hereby incorporated by reference into this specification.

U.S. Pat. No. 6,077,684 is illustrative of some of the Kravtsov technology. This patent, in claim 1 thereof, describes: “A method of determining the anti-leukemic activity of a substance, comprising: a. obtaining a sample of cells from a subject with leukemia; b. isolating a single cell suspension from the sample; c. enriching the sample for leukemic cells by removing non-leukemic cells from the sample; d. placing the enriched leukemic cells in culture; e. exposing a culture of the enriched cells to the substance; f. incubating the cultured cells; g. measuring in a serial manner the optical densities of the culture exposed to the substance; h. measuring in a serial manner the optical densities of a culture of the enriched cells not exposed to the substance; i. subtracting at each serial time point the optical densities of the culture of cells not exposed to the substance from the optical densities of the culture of cells exposed to the substance, so as to obtain a net slope of the serially measured optical densities due to the apoptosis-inducing activity of the substance; j. correlating the slope of a net increase over time in the serially measured optical densities of the cells exposed to the substance with anti-leukemic activity” (see claim 1). As will be apparent, claim 1 of U.S. Pat. No. 6,077,684 describes a process for determining the sensitivity of anti-leukemic agents on leukemia cells.

By comparison, claim 2 of U.S. Pat. No. 6,077,684 describes a process for determining the resistance of leukemia cells to anti-leukemic agents. This claim discloses: “2. A method of determining resistance of leukemic cells to an anti-leukemic substance, comprising: a. obtaining a sample of cells from a subject with leukemia; b. isolating a single cell suspension from the sample; c. enriching the sample for leukemic cells by removing non-leukemic cells from the sample; d. placing the enriched leukemia cells in culture; e. exposing a culture of enriched cells to the substance; f. incubating the cultured cells; g. measuring in a serial manner the optical densities of the culture of enriched cells exposed to the substance; h. measuring in a serial manner the optical densities of a culture of the enriched cells not exposed to the substance; i. subtracting at each serial time point the optical densities of the culture of cells not exposed to the substance from the optical densities of the culture of cells exposed to the substance, so as to obtain a net slope of the serially measured optical densities due to the apoptosis-inducing activity of the substance; j. correlating the absence of a net increase or the presence of a reduced slope of a net increase over time in the optical densities of the culture exposed to the substance with resistance to the substance.”

By way of further comparison, claim 3 of U.S. Pat. No. 6,077,684 describes a process for determining the relative activity of anti-leukemic agents on leukemia cells. This claim describes: “3. A method of determining the relative potential effectiveness of a substance for use in anti-leukemic therapy for a selected subject having leukemia, comprising: a. obtaining a sample of cells from the subject with leukemia; b. isolating a single cell suspension from the sample; c. enriching the sample for leukemic cells by removing non-leukemic cells from the sample; d. placing the enriched leukemic cells in culture; e. exposing a culture of the enriched cells to a first selected substance or mixture of the first selected substance and other substances; f. exposing a culture of the enriched cells to a second selected substance or mixture of the second selected substance and other substances; g. incubating the cultured cells; h. measuring in a serial manner the optical densities of the cultures of enriched cells exposed to the first and second substances or mixtures of substances; i. measuring in a serial manner the optical densities of a culture of the enriched cells not exposed to a substance; j. subtracting at each serial time point the serially measured optical densities of the culture of cells not exposed to the substance from the optical densities of the culture of cells exposed to the first substance or mixture of substances and the optical densities of the culture of cells exposed to the second substance or mixture of substances, so as to detect differences in the net slopes of the serial optical densities due to differences in the apoptosis-inducing activity of the first and second substances or mixtures of substances; k. correlating the greater slope of a net increase over time in the serial optical densities of the culture of cells exposed to the first substance compared to the slope of a net increase over time in the serial optical densities of the culture of cells exposed to the second substance with the greater potential effectiveness of the first substance or mixture of the first substance and other substances in anti-leukemic therapy.”

The claims of U.S. Pat. No. 6,077,684 are limited to processes involving anti-leukemic agents. By comparison, the claims of U.S. Pat. No. 6,258,553 relate to agents that induce apoptosis. Claim 1 of this patent describes: “A method of determining the apoptosis-inducing activity of a substance, which comprises: a) measuring the optical density of a first cell culture at more than one time point, wherein the first cell culture was contacted with the substance; b) measuring the optical density of a second cell culture at more than one time point, wherein the second cell culture was not contacted with the substance; and c) determining a net slope, which is the difference between the optical density change over time of the first cell culture and the optical density change over time of the second cell culture; wherein a positive net slope indicates apoptosis-inducing activity of the substance.”

The process described in the Kravtsov patent publications is not adapted to either detect or diagnose or prepare therapy for or to monitor clonal cell populations. It is an object of one embodiment of this invention to provide a process for detecting, diagnosing, and preparing therapy for clonal cell populations.

Referring again to FIG. 1, and in step 26 thereof, one or more samples are obtained from a biological organism. This step 26 may be conducted at the time steps 12 and/or 14 are conducted, or thereafter, or before.

The additional material collected from the biological organism in step 26 may be, e.g., serum, cells that are not diseased (such as, e.g., somatic cells, lymphocytes, granulocytes, dendritic cells, and cytotoxic T lymphocytes (CTL)), and the like. In one particular embodiment, lymphocytes, granulocytes, dendritic cells, and CTLs are collected from the peripheral blood of an individual patient and are used as controls for toxicity and chemosensitivity testing of an individual patient's normal cells, e.g. “non-cancerous” cells, to assess the risk of life-threatening toxicity if a particular drug combination is administered to the patient. One may use conventional means to assess toxicity. Thus, e.g., one may use the toxicity evaluation processes described in U.S. Pat. No. 5,736,352 (method and apparatus for determination of the activity of cholesterol oxidase and method and apparatus for evaluation of the toxicity of chemical substances), U.S. Pat. No. 6,878,518 (methods for determining steroid responsiveness), and U.S. Pat. No. 6,878,522 (methods for the identification of compounds useful for the treatment of disease states mediated by prostaglandin D2). The entire disclosure of each of these United States patents is hereby incorporated by reference into this specification.

In another embodiment, not shown, the lymphocytes, granulocytes, dendritic cells, and CTLs collected from the peripheral blood of a patient in step 26 are used to compare the molecular character of normal cells to that of the diseased cells of the particular patient.

In another embodiment, not shown, the lymphocytes, dendritic cells, and the like, collected from the peripheral blood of a patient in step 26 are used to allow assessment of the capacity of that individual patient to mount an immune response to a given antigen. In one preferred embodiment, the assessment will be used to indicate the likelihood of an immunotherapeutic response. Additionally, or alternatively, one may collect clinical information (from a clinical laboratory) that also may be submitted to database 28.

In one embodiment, the serum of a patient is collected to be used for further analyses. As is known to those skilled in the art, serum is the fluid obtained from blood after it has been allowed to clot; it is also the plasma without fibrogen.

One may use conventional means for collecting the serum from the biological organism. Thus, e.g., one may use one or more of the processes and/or devices disclosed in U.S. Pat. No. 4,775,620 (cytokeratin tumor markers and assays for their detection), U.S. Pat. No. 5,120,413 (analysis of samples using capillary electrophoresis), U.S. Pat. No. 5,159,063 (isolation and characterization of 120 kDa glycoprotein plasma), U.S. Pat. No. 5,259,939 (capillary electrophoresis buffer), U.S. Pat. No. 5,630,924 (compositions, methods, and apparatus for ultrafast electroseparation analyses), and the like. The disclosure of each of these United States patents is hereby incorporated by reference into this specification.

In one embodiment, in step 26 the white blood cells of the biological organism are collected and analyzed. One may make such collection and analyses by conventional means. Reference may be had, e.g., to U.S. Pat. No. 4,187,979, the entire disclosure of which is hereby incorporated by reference into this specification.

Referring again to FIG. 1, data obtained in steps 18 and/or 20 and/or 22 and/or 24 and/or 26 are preferably conveyed via lines 19 and/or 21 and/or 23 and/or 25 and/or 27 to database 28.

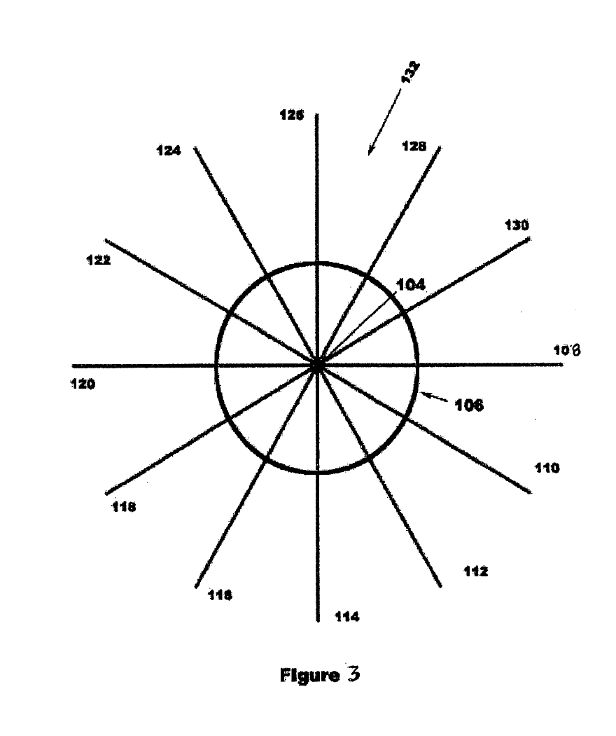

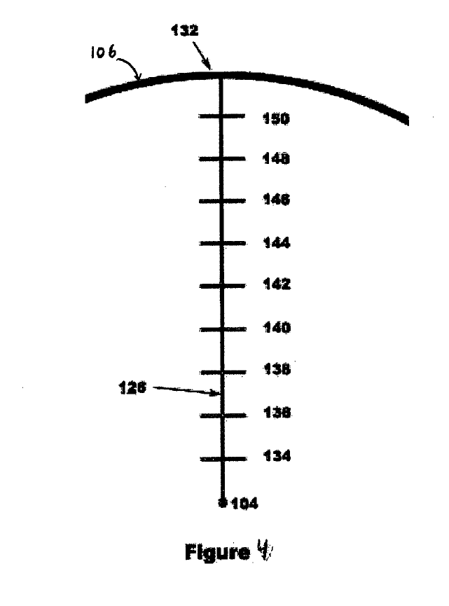

In one embodiment, the database 28 is a relational informatics database in which incoming information is organized according to the “Hankins Medical Mapping System (HaMMS)” and “Hankins coordinates,” as defined below by reference to FIG. 3, which will serve as relational links between samples, diagnoses, treatments, and technologies. The “Hankins Medical Mapping System database” may be constructed in accordance with conventional means disclosed in the prior art. Reference may be had, e.g., to U.S. Pat. No. 5,706,498 (gene database retrieval system where a key sequence is compared to database sequences by a dynamic programming device), U.S. Pat. No. 5,970,500 (database and system for determining, storing, and displaying gene locus information), U.S. Pat. No. 6,023,659 (database system employing protein function hierarchies for viewing biomolecular sequence data), U.S. Pat. No. 6,256,647 (method of searching database of three-dimensional protein structures), U.S. Pat. No. 6,532,462 (gene expression and evaluation system using a filter table with a gene expression database), and the like. The entire disclosure of each of these United States patents is hereby incorporated by reference into this specification.

By way of illustration and not limitation, U.S. Pat. No. 5,970,500 describes and claims “1. A method of displaying the genetic locus of a biomolecular sequence, the method comprising the following: providing a database including multiple biomolecular sequences, at least some of which represent open reading frames located along a contiguous sequence on an organism's genome; identifying a selected open reading frame; and displaying the selected open reading frame together with adjacent open reading frames located upstream and downstream from said selected open reading frame, wherein the adjacent open reading frames and the selected open reading frame are displayed in the relative positions in which they occur on the contiguous sequence.”

FIG. 3 is a schematic of the first two dimensions of the “Hankins Medical Mapping System” 100 that allows kinetic mapping of life, life's molecules, and life's processes. This system is preferably derived from the data produced in steps 18 and/or 20 and/or 22 and/or 24 and/or 26. The “Hankins Medical Mapping System” 100 fully consists of 5 dimensional representations as is described elsewhere in this specification. These 5 dimensions allow a complete kinetic mapping of a cell, the cell's molecules, the cell's processes, and the cell's responses to agents in its environment.

Referring again to FIG. 3, the “Hankins Medical Mapping System” 100, in the preferred embodiment depicted, is in the form of a unit circle 106 with its center 104 at the origin of a polar coordinate system. The system depicts the biological cycle of cell differentiation along myriad vectors or radii from a zygote or stem cell 104 to a fully differentiated cell. At the origin of 104 of the medical mapping system is the zygote or stem cell, from which any number of differentiation vectors may radiate. The magnitude of a given vector, which may be less than or equal to 1, describes the extent to which a particular cell lineage has progressed towards full differentiation. The angle of the vector describes the order of the represented cell lineage in the overall progression of the differentiation of the organism: the larger the angle, the later in the progression the given lineage develops.

Referring again to FIG. 3, and as will be apparent to those skilled in the art, the components of coordinate system are not drawn to scale for purposes of ease of illustration.

Referring again to FIG. 3, it will be seen that, in the preferred embodiment depicted, and connected to center 104, are there are twelve radii of differentiation 108, 110, 112, 114, 116,118, 120, 122, 124, 126, 128, and 130. In one embodiment, these twelve radii of differentiation are separated from each other by about 30 degrees. In the embodiment depicted, radius 126 represents formation of blood islands, the first cellular evidence of the tissue blood. Radii 128 et seq. represent the next organ systems to develop in the fetal development of the organism. As should be readily apparent, the choice of twelve radii of differentiation was made for purposes of illustration; there often are more than twelve radii which will be present in a complete mapping of all of an organism's cell lineages. The radii present will include but not be limited to, e.g., erythrocytes, granulocytes, B cells, T cells, spleen, liver, brain, etc.

Referring to FIG. 3, it will be seen that each of such radii of differentiation 108 et seq. emanate from the origin 104 (at which the zygote/stem cell is located) and radiate toward the periphery 132. The distance between the origin 104 and the periphery 132 represents the space and time over which a cell differentiates from an immature stem cell to a mature cell capable of performing functions for the biological organism. Each of such radii can be divided into units between 0 and 1 that reflect the degree of differentiation.

FIG. 4 is a schematic illustration of how one of the radii of differentiation, radius 126, may be divided into, e.g., ten distinct units 134, 136, 138, 140, 142, 144, 146, 148, 150, and 132. In the embodiment depicted in FIG. 4, each of the units 134 et seq. reflects a percentage of the extent to which the development process in question, from the egg/sperm cell has neared completion (at point 132). These units in toto reflect the span of the cell, from origin to death.

Without wishing to be bound to any particular theory, applicant believes that, during the development of a cell, such cell will trace its lineage along a particular line beginning at origin, 104, and developing along a particular line out to the unit circle, 106. As cells develop, they progress as a family or clone of cells in only one direction and remain on a particular line. Knowledge of where a cell is in its particular developmental pathway will provide the necessary information to allow a physician the ability to promote growth of the cell or to terminate the growth of the cell.

In one embodiment, not shown, gene expression can be documented at each of the different radii of differentiation. As is known to those skilled in the art, gene expression is a multistep process, and regulation of the process, by which the product of a gene is synthesized.

As will be apparent to those skilled in the art, the coordinate system depicted in FIG. 3, which is analogous to polar coordinates, can be used to construct a polar map of gene expression, protein expression, drug responsiveness, polymorphisms, single point mutations, additions, and deletions, physiological processes, and the like.

In one embodiment, not shown, a vector rising in the z-coordinate in the base cylindrical medical mapping coordinate system from a point disposed approximately midway in said vector is used to document gene expression. As a cell progresses along its particular developmental pathway (i.e. lineage), different genes will be expressed. When the gene expression, which was observed in Step 20 and/or Step 22 in FIG. 1, is entered into the database 28, the gene expression can be used as a reference point to document a cells' location along its particular developmental pathway.

In the aforementioned embodiment, for the cell which is at such midway point, the genes which are being expressed may be referenced as other first discrete points, and the genes which are not being expressed may be referenced as additional discrete points. Genetic expression, as evidenced e.g. by the functioning receptors on the cell membrane surface and the proteins being generated by the cell and the like, may thus be represented. As a particular cell or group of cells progresses through its development and matures, different genes will be expressed. The genetic expression of the cell is readily observable and may be used as a marker to identify the location of the cell along its developmental pathway. Thus as a cell traverses its specific lineage pathway the degree of gene expression for a particular gene will vary. In one particular embodiment, not shown, the particular cell is an erythrocyte precursor destined to make an erythrocyte and the erythropoietin receptor expression will be at a certain percentage, e.g. 50 percent, of its maximal level of expression relative to certain housekeeping genes, e.g. actin, gap dehydrogenase, and the like, and the globin expression will be at a certain percentage, e.g. 5 percent, of its maximal level of expression relative to the reference housekeeping genes.

In another embodiment, also not shown, a vector rising in a fourth coordinate in the base cylindrical medical mapping coordinate system from at a discrete point and a vector rising from the discrete point are used to document normal or abnormal gene expression. In one embodiment, not shown, for the cell which is at point 140 in FIG. 4 a gene or genes which is/are expressed at a discrete point or points can be shown as being expressed in a normal non-mutated manner. Additionally, an additional gene or genes which is/are expressed at an additional discrete point or points can be shown as being expressed in a mutagenic cancerous manner.

Without wishing to be bound to any theory, applicant believes that sequences deviating from normal may result from somatic point mutations and/or single nucleotide polymorphisms, and/or chromosomal deletions, additions and/or translocations.

In another embodiment, also not shown, a particular cell or group of cells at a particular developmental address is responsive to exposure to various external agents such as chemotherapy drugs, hormones, or other biologicals, radiation or infectious agents, and the like. In one particular embodiment, also not shown, the cell is a chronic myelogenous leukemia (CML); as such the cell is responsive to hemopoietic lineage specific hormones, e.g., erythropoietin, but is not responsive to non-hemopoietic lineage specific hormones, e.g. estrogen.

In another embodiment, also not shown, the cell is an ovarian cancer cell; as such the cell is responsive to ovarian lineage specific hormones, e.g. estrogen, follicle stimulating hormone, and the like, but it is not responsive to non-ovarian lineage specific hormones, e.g. thyrotropin, erythropoietin, and the like.

FIG. 5 illustrates a process 14 (see FIG. 1) for preserving, expanding and further analyzing the physiology or pathophysiology of the tissue sample obtained in step 12 (see FIG. 1) in live or viable state. The process of step 14 of FIG. 1 may comprise the steps of freezing cells (in step 200), and/or constructing a molecular bank of the cells (in step 202), and/or vitrification of the cells (in step 204), and/or constructing primary cell lines (in step 206), and/or using a “scid mouse” (in step 208).

In step 200, the tissue sample may be preserved by freezing it and its cells. This process may be effected by conventional means. Reference may be had to, e.g., U.S. Pat. No. 5,102,783 (composition and method for culturing and freezing cells and tissues), U.S. Pat. No. 5,958,670 (method of freezing cells and cell-like materials), U.S. Pat. No. 6,140,123 (method for conditioning and cryopreserving cells), and the like. The entire disclosure of each of these United States patents is hereby incorporated by reference into this specification.

In step 202, a molecular bank of the cells may be constructed by conventional means. Reference may be had, e.g., to U.S. Pat. No. 4,849,349 (genes for biologically active proteins), U.S. Pat. No. 5,308,770 (cloning and overexpression of glucose-6-phosphate dehydrogenase from Leuconostoc dextranicanus), U.S. Pat. No. 5,656,467 (methods and materials for producing gene libraries), U.S. Pat. No. 5,869,295 (methods and materials for producing gene libraries), U.S. Pat. No. 6,310,191 (generation of diversity in combinatorial libraries), and the like. The entire disclosure of each of these United States patents is hereby incorporated by reference into this specification.

In one embodiment, in step 202, a gene library is prepared. As is known to those skilled in the art, a gene library is a clone library that contains a large number of representative nucleotide sequences from all sections of the DNA of a given genome; it is a random collection of DNA fragments from a single organism, linked to vectors, and cloned in a suitable host. The DNA from the organism of interest is fragmented (enzymatically or mechanically), the fragments are linked to suitable vectors (plasmids or viruses), the modified vectors are introduced into host cells, and the latter are cloned. A gene library contains both transcribed DNA fragments (exons) as well as nontranscribed fragments (introns, spacer DNA). Retrieval of specific DNA sequences from a gene library frequently involves screening by means of a probe. Reference may be had, e.g., to U.S. Pat. No. 4,874,845 (T lymphocyte receptor subunit), U.S. Pat. No. 4,966,846 (molecular cloning and expression of a vibrio proteolyticus neutral protease gene), U.S. Pat. No. 5,252,475 (methods and vectors for selectively cloning exons), U.S. Pat. No. 5,721,110 (methods and compositions useful in the diagnosis and treatment of autoimmune diseases), U.S. Pat. No. 6,054,267 (method for screening for enzyme activity), U.S. Pat. No. 6,291,161 (method for tapping the immunological repertoire), U.S. Pat. No. 6,472,146 (methods for identification on internalizing ligands and identification of known and putative ligands), U.S. Pat. No. 6,613,528 (cellulose films for screening), U.S. Pat. No. 6,555,315 (screening for novel bioactivities), and the like. The entire disclosure of each of these United States patents is hereby incorporated by reference into this specification.

In one embodiment, and referring again to step 202, a cDNA library is prepared. As is known to those skilled in the art, a cDNA library is a clone library that differs from a gene library in that it contains only transcribed DNA sequences (exons) and no nontranscribed DNA sequences (introns, spacer DNA). It is established by making complementary DNA from a population of cytoplasmic mRNA molecules, using the enzyme RNA-dependent DNA polymerase (reverse transcriptase), converting the single-stranded cDNA to double-stranded DNA, and cloning the latter as in the establishment of a gene library. Reference may be had, e.g., to U.S. Pat. No. 5,700,644 (identification of differentially expressed genes), U.S. Pat. No. 6,143,528 (method for forming full-length cDNA libraries), U.S. Pat. No. 6,174,669 (method for making full-length cDNA libraries), U.S. Pat. No. 6,221,585 (method for identifying genes underlying defined phenotypes), U.S. Pat. No. 6,607,899 (amplification-based cloning method), and the like. The entire disclosure of each of these United States patents is hereby incorporated by reference into this specification.

Referring again to FIG. 5, and in step 204 thereof, one may preserve the tissue sample and its cells by the process of vitrification. As is know to those skilled in the art, vitrification is an experimental procedure for preserving human organs in which chemicals are added prior to cooling to prevent crystallization of water within and outside the cells, so that, with cooling, the molecules essentially become fixed in place. Reference may be had, e.g., to U.S. Pat. No. 4,559,298 (cyroperservation of biological materials in a non-frozen or vitreous state), U.S. Pat. No. 5,200,399 (method of protecting biological material from destructive reactions in the dry state), U.S. Pat. No. 5,290,765 (method for protecting biological materials form destructive reactions in the dry state), U.S. Pat. No. 5,518,878 (cryopreservation of cultured skin or cornea equivalents with agitation), U.S. Pat. No. 5,962,214 (method of preparing tissues and cells for vitrification), U.S. Pat. No. 6,500,608 (method for vitrification of biological cells), U.S. Pat. No. 6,519,954 (cryogenic preservation of biologically active material using high temperature freezing), and the like. The entire disclosure of each of these United States patents is hereby incorporated by reference into this specification.

Referring again to FIG. 5, in step 206 thereof, primary cell lines are prepared by conventional means. As is known to those skilled in the art, a primary culture is a culture that is started from cells, tissues, or organs that are derived directly from an organism, or tissue freshly explanted from the organism. Reference may be had, e.g., to U.S. Pat. No. 5,399,493 (methods and compositions for the optimization of human hematopoietic progenitor cell cultures), U.S. Pat. No. 5,437,994 (method for the ex vivo replication of stem cells, for the optimization of hematopoietic progenitor cell cultures, and for increasing the metabolism, GM-csf secretion, and/or IL-6 secretion of human stromal cells), U.S. Pat. No. 5,474,770 (biological support for cell cultures constituted by plasma proteins coagulated by thrombin, its use in the preparation of keratocyte cultures, their recovery and their transport for therapeutic purposes), U.S. Pat. No. 5,602,028 (system for growing multi-layered cell cultures), U.S. Pat. No. 5,658,797 (device for the treatment of cell cultures), U.S. Pat. No. 5,728,541 (method for preparing cell cultures from biological specimens for chemotherapeutic and other assays), U.S. Pat. No. 5,888,816 (cell cultures of and cell culturing method for nontransformed pancreatic, thyroid, and parathyroid cells), and the like. The entire disclosure of each of these United States patents is hereby incorporated by reference into this specification.

In one embodiment of the invention, it is preferred to culture tumor cells by defining the growth requirements (hormones, growth factors) that select for and propagate the tumor cells and not contaminating fibroblast and other non-tumor stromal cells.

Referring again to FIG. 5, and to step 208 thereof, cells from the tissue sample are implanted into an immunodeficient mouse. One may use any of the immunodeficient mice known to the prior art in this step 208 (Severe Combined Immunodeficient, SCID, or Non Obese Diabetic-SCID, NOD-SCID mice). Reference may be had, e.g., to U.S. Pat. No. 5,602,305 (immunodeficient animal model for studying T cell-mediated . . .), U.S. Pat. No. 5,625,127 (extended humanhematopoiesis in a heterologous host), U.S. Pat. No. 5,633,426 (in vivo use of human bone marrow for investigation and production), U.S. Pat. No. 5,639,939 (chimeric immunocompromised mammal comprising vascularized fetal organ tissue), U.S. Pat. No. 5,643,551 (small animal metastasis model), U.S. Pat. No. 5,849,998 (transgenic animals expressing a multidrug resistance cDNA), U.S. Pat. No. 5,859,307 (mutant RAG-1 deficient animals having no mature B and T lymphocytes), U.S. Pat. No. 5,925,802 (functional reconstitution of SCID-bo mice with bovine fetal hematopoietic tissues), U.S. Pat. No. 5,986,170 (murine model for human carcinoma), U.S. Pat. No. 5,994,617 (engraftment of immune deficient mice with human cells), U.S. Pat. No. 6,087,556 (transgenic animals capable of replicating hepatitis viruses and mimicking chronic hepatitis infection in humans), U.S. Pat. No. 6,284,239 (murine model for human carcinoma), U.S. Pat. No. 6,353,150 (chimeric mammals with human hematopoietic cells), U.S. Pat. No. 6,410,824 (animal model for psoriasis for the prevention and treatment of psoriasis in humans), U.S. Pat. No. 6,509,514 (chimeric animal model susceptible to human hepatitis C virus infection), U.S. Pat. No. 6,620,403 (in vivo chemosensitivity screen for human tumors), and the like. The disclosure of each of these United States patents is hereby incorporated by reference into this specification.

In one embodiment, a “scid mouse” (“severe combined immunodeficient” mouse) is implanted with the cells of the tissue sample. These mice are well known to those in the art and are described, e.g., in U.S. Pat. No. 5,994,617 (engraftment of immune-deficient mice with human cells), U.S. Pat. No. 6,284,239 (murine model for carcinoma), U.S. Pat. No. 6,107,540 (mice models of human prostate cancer progression), 6,639,121 (inducible cancer model to study the molecular basis of host tumor cell interactions in vivo), and the like. The entire disclosure of each of these United States patent applications is hereby incorporated by reference into this specification.

Referring again to FIG. 5, the scid mouse of step 208 may be used for preservation and expansion of the cells (see step 210), and/or tumor modeling (see step 212), and/or serum biomarker analysis (see step 214), and/or the construction of a personalized xenograph (see step 216), and/or hormone requirement analysis (see step 218).

In one embodiment of step 214, purified tumor cells produced in step 16 of FIG. 1 are transplanted in the scid mouse (see step 208 of FIG. 5), the transplanted tumor cells are allowed to grow for a period of up to about one year or more. Serum samples are periodically collected from the implanted mouse, preferably on a monthly basis; and the serum from the transplanted recipient mouse is periodically analyzed by serum proteomics technology. Such serum analysis techniques are well known. By way of illustration, reference may be had, e.g., to U.S. Pat. No. 4,115,062 (cancer analysis by serum analysis of glycolipids), U.S. Pat. No. 5,223,397 (soluble HLA cross-match), U.S. Pat. No. 5,270,169 (detection of HLA antigen-containing immune complexes), U.S. Pat. No. 5,482,841 (evaluation of transplant acceptance), U.S. Pat. No. 6,019,945 (sample analysis system), and the like. The entire disclosure of each of these United States patents is hereby incorporated by reference into this specification.

Referring again to FIG. 5, and to step 214 thereof, in another embodiment, one may use tumor stem cells that are identified, e.g., by the process described in U.S. Pat. No. 5,994,617 of John E. Dick, the entire disclosure of which is hereby incorporated by reference into this specification.

Referring again to FIG. 1, and in the preferred embodiment depicted therein, information from the database 28 may be used to deduce the developmental address of the single cells (in step 30), and/or to deduce the best therapy for treating a disease condition and/or to discover new therapies (in step 32), and/or to deduce a biomarker panel and/or to thus discover new biomarkers (in step 34). The deduction of the developmental address (in step 30) may lead to lineage specific drug discovery (in step 36), and/or a lineage specific response/diagnostic (in step 38), and/or to a lineage specific screening platform (in step 40).

Referring again to FIG. 1, in step 30, the information from the database 28 is used to deduce the developmental address of normal and/or abnormal cells. In one preferred embodiment, the coordinate system depicted in FIG. 3 is used to deduce the developmental address of normal or abnormal cells.

Referring again to FIG. 1, the developmental address of normal and/or abnormal cells can be used to lineage specific drug discoveries (step 36), and/or lineage specific responses and/or diagnostics (step 38), and/or lineage a specific screening platform (step 40).

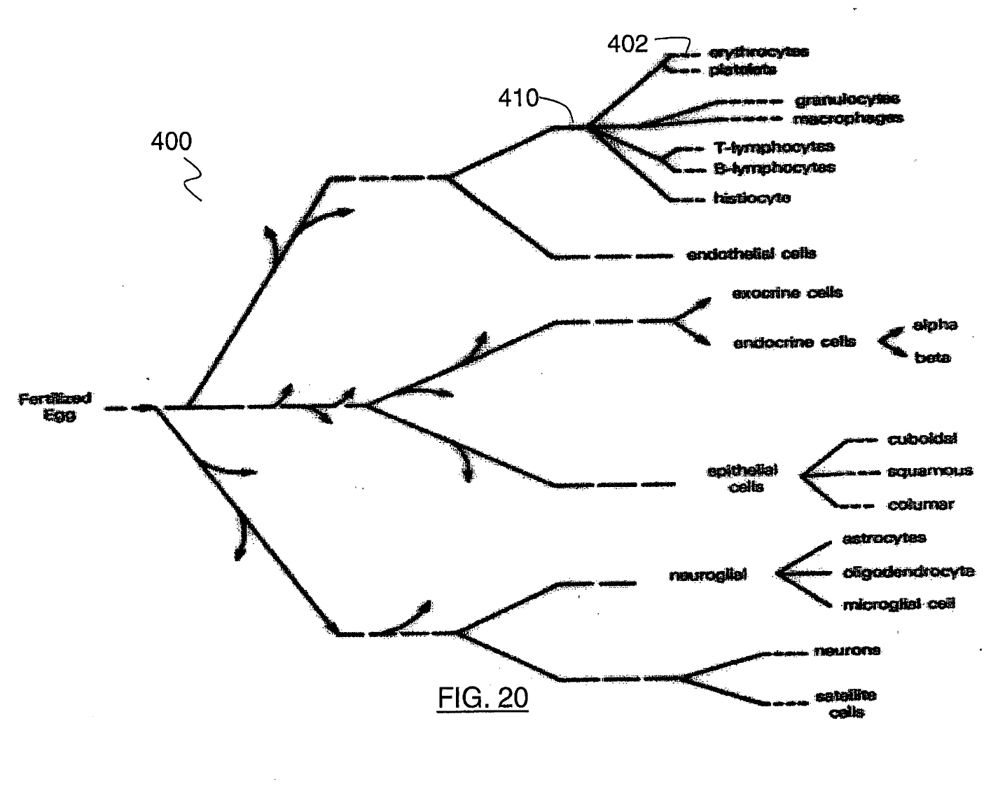

FIG. 20 is a schematic representation of a preferred process 400 for deducing the developmental address of cells that are abnormal. In the preferred embodiment, not shown, the developmental address of abnormal cells is deduced. Abnormal cells maintain four properties of normal cells, viz., hormone sensitivity, the need for specific viability factors to survive, the ability to mature through their lineage pathway, and the exhibition of heterogeneity by the clones of the cells. The observation of these properties is preferred to deduce the developmental address of abnormal cells. The abnormal cells possess a hormone dependence and therefore also require specific viability factors to survive. By way of illustration, if the abnormal cell depicted at reference 402 in FIG. 20 is an erythroleukemia, it will require erythropoietin to survive. If it is a T-cell lymphoid leukemia, it will require interleukin 2 to survive.

By way of further illustration, if the abnormal cell, is a melanoma, it will require a melanocyte lineage specific hormone to survive.

By way of further illustration, if the abnormal cell, not shown, is an ovarian cancer, it will require follicle stimulating hormone to survive.

By way of further illustration, if the abnormal cell, not shown, is FIG. 20 is a thyroid cancer, it will require a thyroid lineage specific factor to survive.

Referring again to FIG. 20 (see element 410), abnormal cells are not blocked from progressing through their specific cell lineage pathway. In one preferred embodiment, not shown, the abnormal cells in question are chronic myeloid leukemia cells exhibiting the Philadelphia chromosome. A normal hemopoietic stem cell would progress along its lineage pathway to produce mature granulocytes, erythrocytes, and the like. The chronic myeloid leukemia cell, in the presence of the proper nutrients and specific viability factors, e.g. erythropoietin, will develop into mature cells.

Referring again to FIG. 20, clones can exhibit heterogeneity. As known to those skilled in the art, a clone is a group of genetically identical cells all descended from a single common ancestral cell by mitosis in eukaryotes or by binary fission in prokaryotes; clone cells also include populations of recombinant DNA molecules all carrying the same inserted sequence. In one preferred embodiment, not depicted herein, the chronic myeloid leukemia cell exhibiting the Philadelphia chromosome will progress along any of three different lineage pathways. This is analogous to the normal hemopoietic stem cell progressing along its lineage pathway to produce mature granulocytes, erythrocytes, and the like. The chronic myeloid leukemia cell will develop into mature cancerous granulocytes, erythrocytes, and the like. Thus the clones of the original chronic myeloid leukemia cell exhibit different characteristics.

Referring again to FIG. 1, in step 32, the developmental address of abnormal and/or normal cells can be used to deduce the best therapy to treat the abnormal cells. In one embodiment, the developmental address of abnormal cells is used to deduce the best therapy to treat the abnormal cells. In one additional embodiment, the developmental address of abnormal and normal cells is used to deduce the best therapy to treat the abnormal cells.

Referring again to FIG. 1, in step 34, the developmental address of normal and/or abnormal cells are used to deduce biomarker panel.

Without wishing to be bound to any theory, applicant believes that the thyroid stimulating hormone (TSH) is required for the viability of thyroid cancer cells and, thus, agents which interfere with TSH hormone and/or its interaction with its receptor will lead to death of thyroid cancer cells.

Without wishing to be bound to any theory, applicant also believes that the FLT-3 ligand is required for the viability of certain acute leukemia cells and, thus, agents which interfere with FLT-3 ligand and/or with the interaction of such ligand with its receptor will lead to the death of such acute leukemia cells.

Again, without wishing to be bound to any particular theory, applicant also believes that the follicle stimulating hormone (FSH) is required for the viability of ovarian cancer cells and, thus, agents which interference with either FSH and/or with the interaction of such hormone with its receptor will lead to the death of ovarian cancer cells.

Additionally, applicant believes that agents that interfere with the ligands for EGF receptor III or EGF receptor IV will result in the death of particular tumor cells which are found to express the genes for these receptors and which display said receptors as a part of the tumor cells.

As is known to those skilled in the art, various agents can interfere with one or more of the aforementioned moieties. Such agents may include, e.g., soluble receptors that compete with the receptors on the cancer cells for the ligand and, after binding with the ligand, may be flushed from a biological system. Such agents may also include, e.g., antibodies against the ligand and/or the receptor including, e.g., antibodies that carry toxic molecules (such as radioactive moieties or cytotoxic moieties). Such agents may also include, e.g., small molecules that bind to the receptor or its ligand and thus compete with the cancer receptor/ligand binding event; such agents also may include antisense molecules that block the synthetic path leading to the receptor at one or more sites, thus leading to the death of the cancer cell.

Improvement upon the Process of U.S. Pat. No. 6,258,553

In this section of the specification, an improvement upon the process described in U.S. Pat. No. 6,258,553 is presented.

U.S. Pat. No. 6,258,553 has two independent claims, claims 1 and 2. Claim 1 of this patent describes: “1. A method of determining the apoptosis-inducing activity of a substance, which comprises: a) measuring the optical density of a first cell culture at more than one time point, wherein the first cell culture was contacted with the substance; b) measuring the optical density of a second cell culture at more than one time point, wherein the second cell culture was not contacted with the substance; and c) determining a net slope, which is the difference between the optical density change over time of the first cell culture and the optical density change over time of the second cell culture; wherein a positive net slope indicates apoptosis-inducing activity of the substance.” claim 2 of this patent describes: “2. A method of determining resistance of cells to the apoptosis-inducing activity of a substance, comprising: a) measuring the optical density of a first cell culture at more than one time point, wherein the first cell culture was contacted with the substance; b) measuring the optical density of a second cell culture at more than one time point, wherein the second cell culture was contacted with the substance and is apoptotically sensitive to the substance; and c) determining a net slope, which is the difference between the optical density change over time of the first cell culture and the optical density change over time of the second cell culture; wherein a positive net slope indicates resistance of the first cell culture to the apoptosis-inducing activity of the substance.”

At column 7 of U.S. Pat. No. 6,258,533, the term “optical density,” as used in such patent, is defined. It is stated that “The step of measuring optical density of the culture is done by measuring absorbance at about 550 to 650 nanometers. The optical densities of the cultures are preferably read after shaking.”

Thus, as the term “optical density” is used in U.S. Pat. No. 6,258,533, it refers to a measurement of absorbance; and the values described in, e.g., the Figures of such patent appear to be absorbance measurements using a light source with a wavelength of from about 550 to about 650 nanometers.

Applicant has discovered that, when he measures the transmittance rather than the absorbance of the “first cell culture” and the “second cell culture,” a more accurate representation of the actual “net slope” is obtained. Without being bound to any particular theory, applicant believes that the “net slope” indicated by the transmittance values is a more sensitive indication of apoptosis than is the “net slope” indicated by the absorbance values.

As is used in this specification, the term transmittance is the ratio of the radiant power transmitted by an object to the incident radiant power; and it may be measured by conventional means. Reference may be had, e.g., to U.S. Pat. No. 4,019,819 (optical property measurement and control system), U.S. Pat. No. 4,159,874 (optical property measurement system and method), U.S. Pat. No. 4,243,319 (optical property measurement system and method), U.S. Pat. No. 4,288,160 (optical property measurement system and method), U.S. Pat. No. 4,296,319 (watermark detection), U.S. Pat. No. 5,175,199 (high transparency silica-titania glass beads, method for making, and light transmission epoxy resin compositions), U.S. Pat. No. 5,223,437 (direct fibrinogen assay), U.S. Pat. No. 5,670,375 (sample card transport method for biological sample testing machine), U.S. Pat. No. 5,587,795 (self-aligning substrate transmittance meter), U.S. Pat. No. 5,888,455 (optical reader and sample card transport stations for biological sample testing machine), U.S. Pat. No. 5,923,039 (ultraviolet transmittance analyzing method and instrument), U.S. Pat. No. 5,971,537 (lens specifying apparatus), U.S. Pat. No. 6,040,913 (method to determine light scattering efficiency of pigments), U.S. Pat. No. 6,236,460 (method for determining the light scattering efficiency of pigments), U.S. Pat. No. 6,320,661 (method for measuring transmittance of optical members), and the like. The entire disclosure of each of these United States patents is hereby incorporated by reference into this specification.

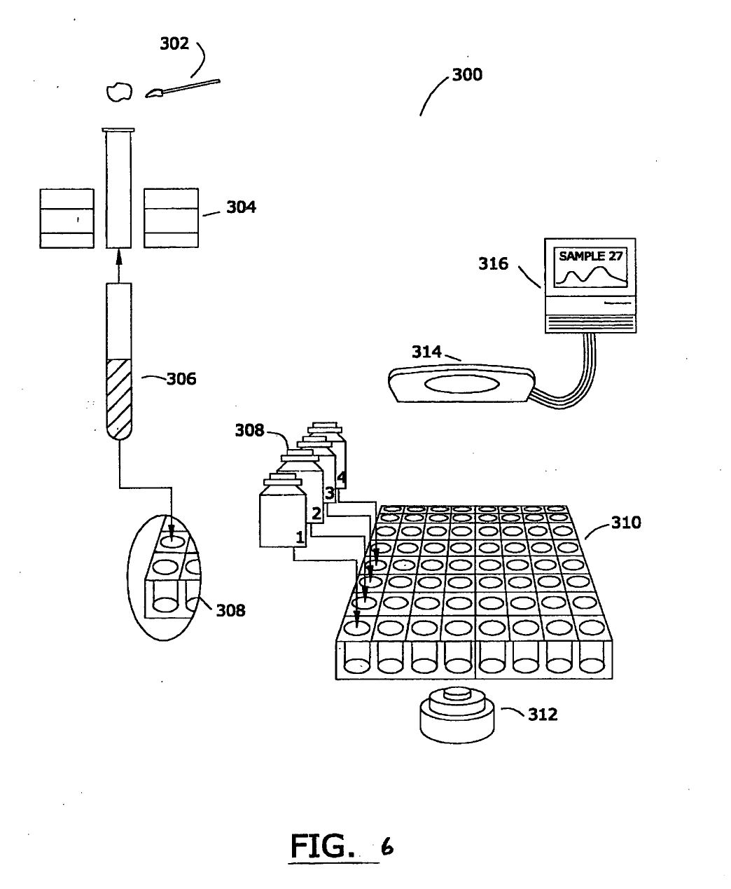

FIG. 6 is a schematic of one preferred process 300. In the embodiment depicted, and in step 302 thereof, a tissue sample is removed by conventional means. One may use, e.g., the cell procurement method described at lines 50 et seq. of Column 14 of U.S. Pat. No. 6,258,553. As is disclosed in such patent, and by way of illustration and not limitation, “Prior to any chemotherapy, a sample of venous blood (e.g., 1-30 ml) or a sample of bone marrow (e.g., 2-20 m: is obtained by direct needle aspiration under sterile conditions. The samples are drawn into a heparinized syringe and diluted with RPMI-1640 medium that contains phenol red. The mononuclear fraction of each sample is isolated by centrifugation using Ficoll-Hypaque. If erythrocytes contaminate the mononuclear cell fraction, then they are removed by treatment with red cell lysis buffer. After washing three times in phosphate buffered saline, an aliquot of the mononuclear cells is analyzed by either light microscopy or flow cytometry for purity and viability. The specific MAb's that recognized the leukemia cells in the diagnostic testing are used to check purity while 7-amino-actinomycin D (7AAD) is used to check viability. If purity and viability are both greater than 90%, then the cells are aliquoted for the present assays and for cryopreservation in RPMI-1640 containing 20% feta. bovine serum and 10% dimethylsulfoxide. Greater than 90% purity and viability would be expected in most cases with a high leukemic cell count in either the blood or bone marrow. If the mononuclear cell fraction purity is less than 90%, then the cells are further purified. T-lymphocytes and monocytes are removed by negative selection using immunomagnetic separation. MAb's to CD2 for T-cell removal and CD14 for monocyte removal and Dynabeads (Dynal, Inc.) are used in those cases in which the diagnostic immunophenotyping shows that the leukemic cells lack these surface antigens. After these immunomagnetic separations, the leukemic cell population will again be tested for purity.”

Referring again to FIG. 6, and in step 304 thereof, a sample is prepared of the cells from the tissue obtained in step 302. This sample may be prepared by conventional means. Thus, e.g., and referring again to U.S. Pat. No. 6,258,553, “Purified leukemic cells are resuspended at from 1.0×105 to 4.0×105 cells/ml in RPMI-1640 medium without phenol red and with 10% fetal calf serum. Note that depending on the microwell plate and the O.D. reader, the concentration of cells may be significantly lower. Aliquots of 250 μliters are cultured in individual wells of a 96-well, flat-bottomed tissue culture microplate.”

In steps 306/308 of the process, the cell samples are placed in specially prepared culture media to which various agents may have been added. By way of illustration, and referring again to U.S. Pat. No. 6,258,553, “Various concentrations of chemotherapeutic agents used to treat acute leukemias are added to duplicate cultures immediately prior to incubation at about 37° C. in 5% CO2 in humidified air. The ranges of concentrations of the agents are based on a) previous reports of apoptosis induced in vitro by these specific agents in either fresh human leukemia cells or human leukemia cell lines and b) pharmacokinetic studies demonstrating that these ranges include concentrations of the parent drugs and/or their active metabolites found in patients following treatment for leukemia. In the present example, the leukemia samples from adults are tested with four agents that are used in their induction and consolidation therapy: 0.1-10.0 μM idarubicin 10,31; 0.01-1.0 μM daunorubicin 11,31; 0.01-10.0 μM cytosine arabinosidel 12,13,32; 0.1-10.0 μg etoposide 11,17,33 and 0.01-1 μM mitoxantrone 16,34,35. For leukemia samples from children, the same concentrations of cytosine arabinoside and etoposide as listed above for adult samples are examined. In place of idarubicin in the adults, daunorubicin at concentrations of 0.01-1 μM are tested. Control wells will receive an equal volume of solvent used for each chemotherapeutic agent. After 30 minutes incubation in humidified air plus 5% CO2, 60 μliters of sterile light mineral oil is layered over each culture, the microplate covered with a lid, and placed in the incubated microplate reader. The O.D. at 600 nm (590-650 nm) of each culture is monitored every five minutes over the ensuing 48 hour period.”

U.S. Pat. No. 6,258,553 discloses that, prior to having their absorbances determined, the cell cultures are agitated. At lines 47-48 of Column 15 such patent, it is disclosed that: “The cultures are shaken with the mixing mode of the incubated microplate reader before each reading is made.” Similarly, at lines 40-44 of Column 7 of U.S. Pat. No. 6,258,553, it is disclosed that “The step of measuring the optical density of the culture is done by measuring absorbance at about 550 to 650 nanometers. The optical densities of the cultures are preferably read after shaking.”

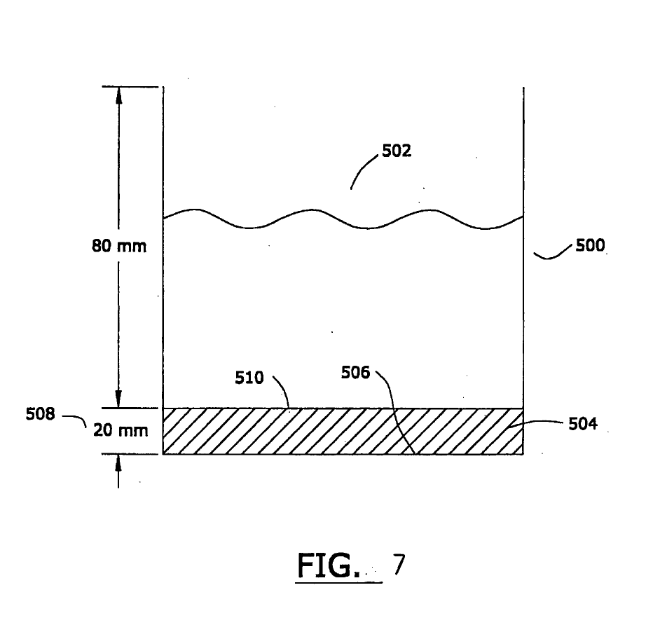

Applicant has discovered that he may provide an improved process by measuring transmittance of cultures that are quiescent rather than agitated. This is illustrated in FIG. 7.

FIG. 7 is a sectional view of a well 500 in which is disposed a culture media 502 that preferably is in a relatively quiescent state. As used herein, the term “relatively quiescent state” means that at least about 90 weight percent of the cellular particulate matter 504 is disposed on the bottom surface 506 of the well and within about the first 20 millimeters distance 508 from such bottom surface 506. Put another way, such wells are typically about 10 centimeters deep, and no more than about 10 weight percent of the cellular particulate matter 504 in the well is disposed above the 20 millimeter line.

Without wishing to be bound to any particular theory, applicant believes that the use of a quiescent state culture medium provides more meaningful data that is more likely to reflect the presence or absence of apoptosis in the cell samples. This is unexpected in view of the clear teaching of U.S. Pat. No. 6,258,553 that a non-quiescent cell culture be used.

Referring again to FIG. 6, and in the preferred embodiment depicted therein, a 96 well microtiter dish 310 is preferably used, and a light source 312 shines light through the samples disposed in such dish. The light source preferably provides light with a wavelength of from 200 about 800 nanometers. In one embodiment, the wavelength provided by the light source is from about 300 to about 700 nanometers. In yet another embodiment, the wavelength provided by the light source is from about 340 to about 660 nanometers.

In the embodiment depicted in FIG. 6, light is being transmitted through a sample 27. The transmitted light is detected by sensor 314, and this information is continually transmitted to controller 316.

In one embodiment, a SpectraMax 340 microplate reader is used for the analyses illustrated in FIG. 6. This microplate reader may be purchased, e.g., from GMI, Inc. of 6551 Jansen Avenue, N.E., Suite 202, Albertville, Minn.

One may use other microplate readers such as, e.g., those disclosed in U.S. Pat. No. D404,140 (microplate reader), U.S. Pat. No. 4,892,409 (photometric apparatus for multiwell plates having a positionable lens assembly), U.S. Pat. No. 5,766,875 (metabolic monitoring of cells in a microplate reader), U.S. Pat. No. 5,784,152 (tunable excitation and/or tunable detection microplate reader), and the like. The entire disclosure of each of these United States patents is hereby incorporated by reference into this specification.

Referring again to FIG. 6, and in the preferred embodiment depicted therein, the transmittance values measured by sensor 314 may be converted into optical density values in accordance with the formula: D_{w}=-log₁₀T_{w}, wherein T_{w} is the measured transmittance of the sample measured at the specified wavelength (w) of the light used, and D_{w} is the optical density of the sample at the specified wavelength of the light used.

As will be apparent to those skilled in the art, although the U.S. Pat. No. 6,258,553 claims to be measuring the optical density of a sample, what in fact it clearly is measuring is only the absorbance of such sample at a specified wavelength. One cannot determine the optical density of a sample merely by measuring the degree to which it absorbs light of a certain wavelength.

Thus, applicant's process allows one to measure the true optical density of a sample rather than mere absorbance. Once such optical density has been measured, one can then plot optical density versus time (as illustrated in the Figures of U.S. Pat. No. 6,258,553) and obtain a true rather than a distorted indication of cell apoptosis.

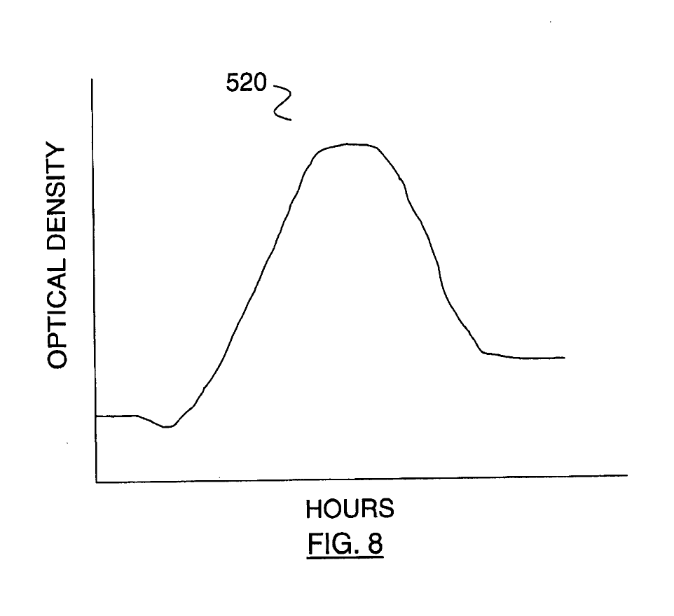

FIGS. 8 through 13 are schematic representation of graphs of data representing the relationships of optical density versus time for various biological systems. In one preferred embodiment, as is shown in FIGS. 8-13 the plots of optical density versus time which are obtained from applicant's process allow one to measure other cell activities besides apoptosis. FIGS. 8 through 13 provide plots of optical density versus time for several cell samples from steps 306/308 from FIG. 6.

By way of illustration, FIG. 8 is a representative graph 520 illustrating cells in the medium undergoing apoptosis, as is evident from the initial increase in optical density during membrane blebbing followed by a decrease in optical density during the breakup of the cells.

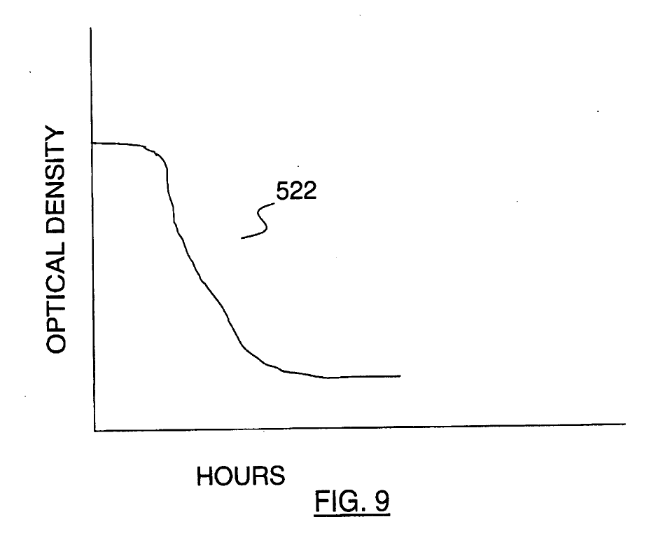

By way of further illustration, FIG. 9 is a representative graph 522 illustrating the behavior of cells in the medium undergoing necrosis, as is evident from the initial decrease in optical density as cells die followed by the optical density remaining constant after there are no more cells to break up.

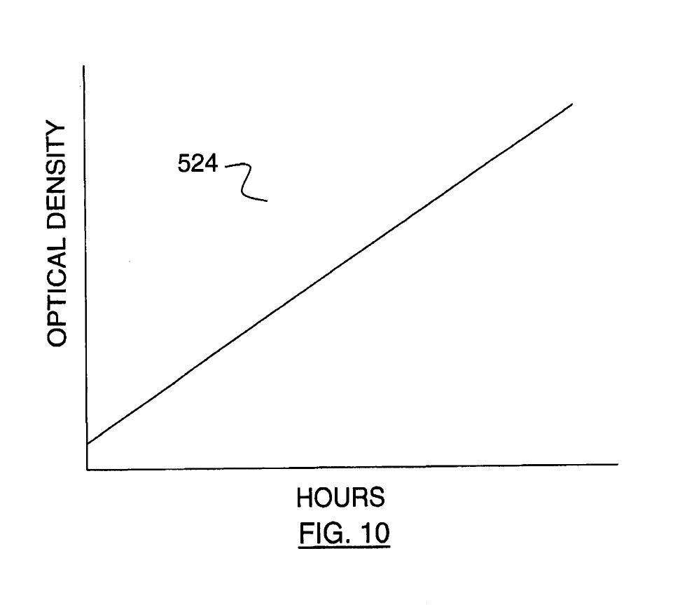

Referring to FIG. 10, this Figure is a representative graph 524 representing cells in the medium undergoing proliferation, as is evident from the continuous increase in optical density which applicant believes is indicative of cell growth and replication within the medium.

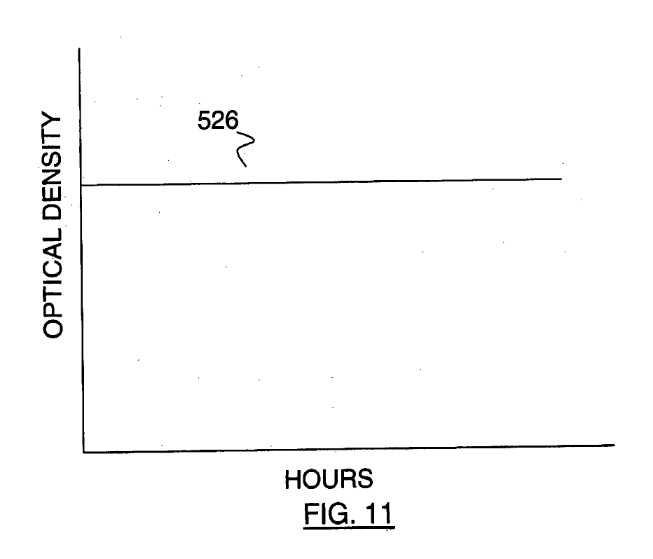

Referring to FIG. 11, graph 526 represents cells in the medium undergoing cytostasis as is evident by the constant nature of the optical density. This is indicative that the cell population within the medium remains constant, i.e., there is neither an appreciable increase nor appreciable decrease in viable cells within the medium.

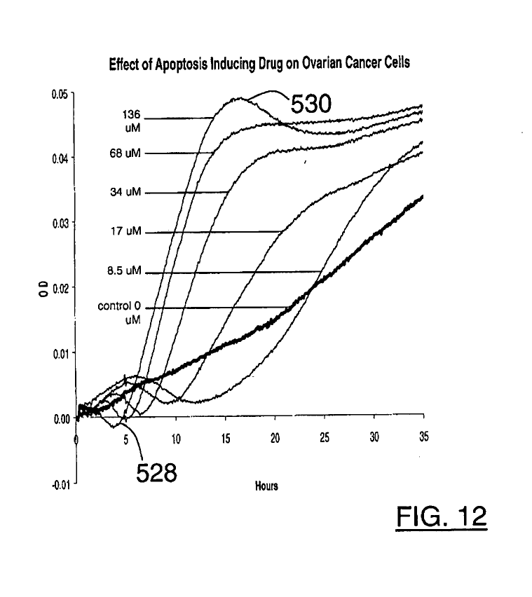

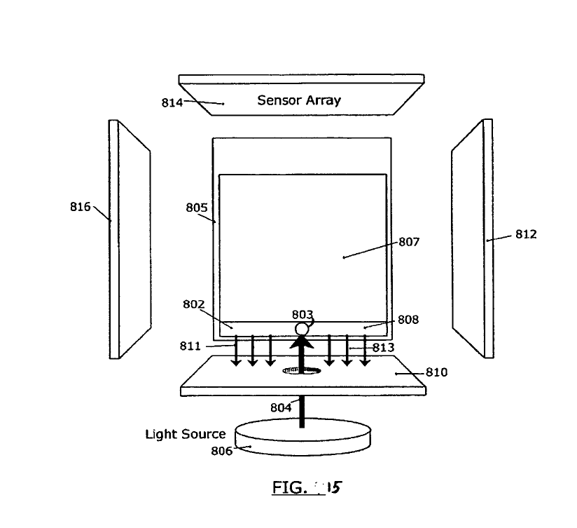

Referring to FIG. 12, a dip 528 in optical density, 528 is observed prior to the development of the “apoptosis peak”. Without wishing to be bound to any particular theory, applicant believes that the dip in optical density at point 528 is due to a decrease in forward light scatter caused by the shrinking of cells, possibly by water loss from the cells prior to the blebbing process. One may arrange the receptor cell(s) 814 in FIG. 15 to kinetically measure only forward scatter. By measuring only the forward scatter, the drug dose dependent dip in optical density may be measured; and one may develop a novel assay for apoptosis that has superiority over any current assays, e.g. a 2 hour assay for cis platin on Ovarian Cancer cells (see point 528 of the graph of FIG. 12).

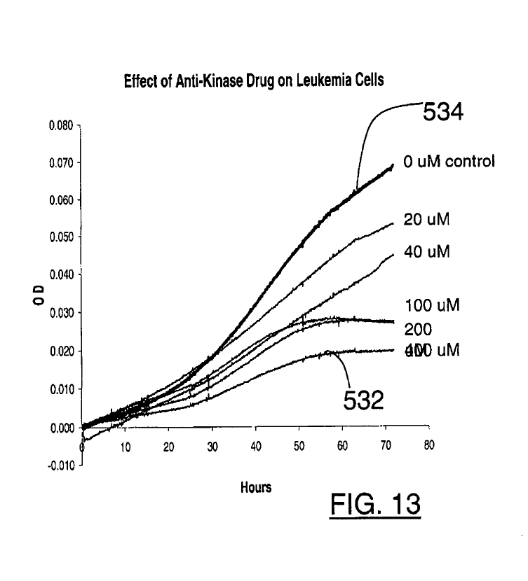

FIG. 13, is a graphical representation of the dose-dependent decrease in measured optical density from the control curve for certain drugs, e.g. imatinib mesylate, which cause apoptosis much more slowly that cytotoxic drugs, e.g. idarubicin. Such drugs do not produce an “apoptosis peak” in the KOR assay. The activity of such drugs can be quantitated by the relative decrease in the experimental slopes, 532 relative to the control slope 534. In one preferred embodiment, shown in FIG. 13, the dose-dependent decrease in slope generated by adding imatinib mesylate, an anti-kinase drug rather than a cytotoxic drug, to K562 tumor cells derived from a patient with chronic myelogenous leukemia.

A Preferred Process Involving Solid Tumors

U.S. Pat. Nos. 6,077,684 and 6,258,553 disclose assays for measuring apoptosis in cell cultures. In the processes described in these patents, cell procurement is conducted by obtaining samples of cells in cell culture media.

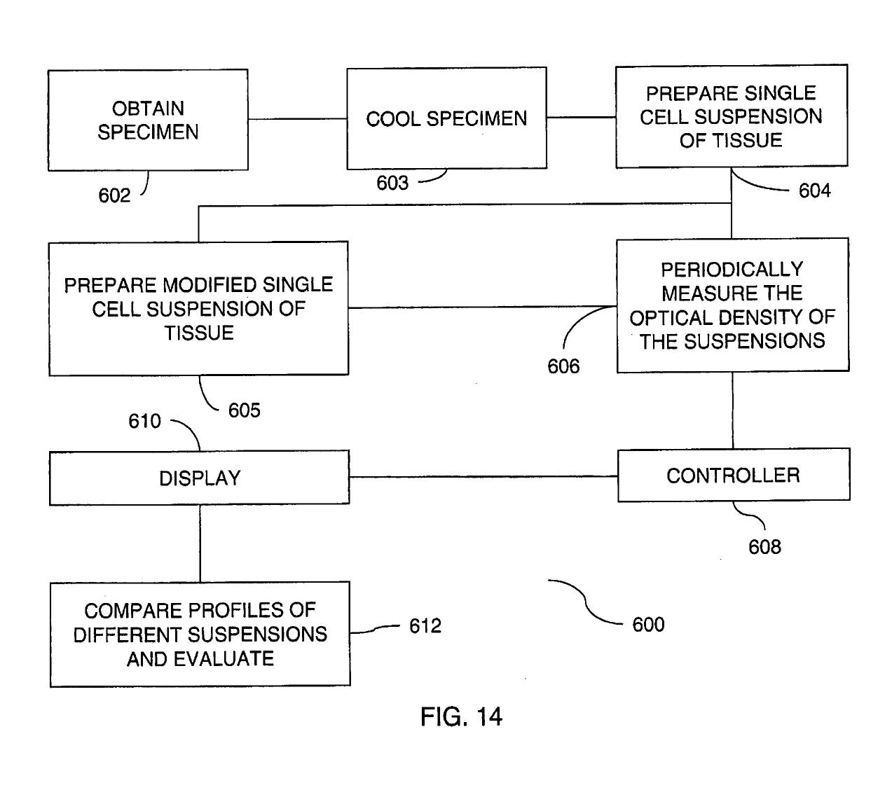

Nowhere in U.S. Pat. Nos. 6,077,684 and 6,258,553 is there any disclosure as to how soon after the cells are procured they are subjected to the specified assay. Applicant has discovered that, in his assay (which utilizes optical density rather than absorbance measurements), it is highly advantageous to use freshly explanted cells in the assay, especially when the cells are derived from solid tumors. This process 600 of conducting this step is illustrated in FIG. 14.

Referring to FIG. 14, and in step 602, a specimen of tissue is obtained in step 602. Such a specimen is often obtained surgically by conventional means. With regard to the remainder of the discussion relating to process 600, it will be assumed that specimen obtained is from a solid tumor; it will be apparent, however, that other sources for the specimen also may be used.

In step 604 of the process, a single cell suspension of the tumor is prepared by conventional means. Thus, e.g., one may use various methods of tissue desegregation such as, e.g., mincing into small pieces. Reference may be had, e.g., to U.S. Pat. No. 5,744,363 (method for establishing a tumor-cell line by preparing single-cell suspension of tumor cells from tumor biopsies), U.S. Pat. No. 6,114,128 (method and kit for predicting the therapeutic response of a drug against a malignant tumor), U.S. Pat. No. 6,448,030 (method for predicting the efficacy of anti-cancer drugs), and the like. The entire disclosure of each of these United States patents is hereby incorporated by reference into this specification.

In one embodiment, the single cell suspension of the tumor cell is preferably prepared within less than about 60 hours of obtaining the tissue sample and, more preferably, within less than about 48 hours of obtaining the sample. In one embodiment, the single cell suspension is prepared from 0.1 to 10 hours after obtaining the tissue sample.

Prior to the time the single cell suspension is prepared, the tissue sample is preferably maintained at a temperature of from about 3 to about 15 degrees Celsius, by cooling (see step 603). It is critical, however, that the tissue sample not be allowed to freeze. In one embodiment, the tissue sample is maintained at from about 4 to about 10 degrees Celsius.

In one embodiment, during each of steps 602, 603, and 604, the source of the specimen and/or the specimen and/or the single cell suspension preferably is exposed to an oxygen-containing gas, such as air.

In another embodiment, during each of steps 602, 603, and 604, the source of the specimen and/or the specimen and/or the single cell suspension is exposed to an oxygen-deficient atmosphere.

In one embodiment, during one or more of the steps 602, 603, and/or 604, the source of the specimen and/or the specimen and/or the single cell suspension is bathed with a solution containing one or more nutrients such as, e.g., glucose, amino acid(s), protein(s), serum, and the like.

In step 606 of the process, the optical density of the cell suspension is periodically measured, as discussed elsewhere in this specification and in U.S. Pat. No. 6,258,553 and 6,077,684.

In step 605 of the process, which may be optional, one may prepare other “modified” single cell suspensions that vary from the suspension 604 in that they contain additional agents, or different agents, or different cells, etc. Thus, e.g., different cell suspensions may contain different concentrations of different chemotherapeutic agents and/or different growth factors and/or different concentrations of such agents and/or factors and/or different combinations of such agents and/or factors. By testing a multiplicity of such combinations, the optimal therapy for a particular malignant tissue may be determined. Alternatively, the optimal growth conditions for at tumor may be determined and thus, lead to means for preventing such growth conditions.

The optical densities of these other, “modified cell suspensions” also are preferably periodically measured in, e.g., a microtiter culture dish assembly. This information is preferably continually fed to controller 608, which continually preferably generates optical density profiles of each of the samples. on a display 610. In one embodiment, the instantaneous changes thus displayed provide information on, e.g., the time when one should add growth agents.

These multiple profiles will enable one to determine when one or more agents should be added, whether one or more agents should be added, the sequence of adding one or more agents, the optimal concentrations and combinations of such agents, and the time course of events subsequent to the addition. This may be done in step 612, where a comparison of profiles made of cell suspensions under different conditions and/or of cell suspensions under similar conditions but with different agents, may be made.

Another Preferred Assay Process

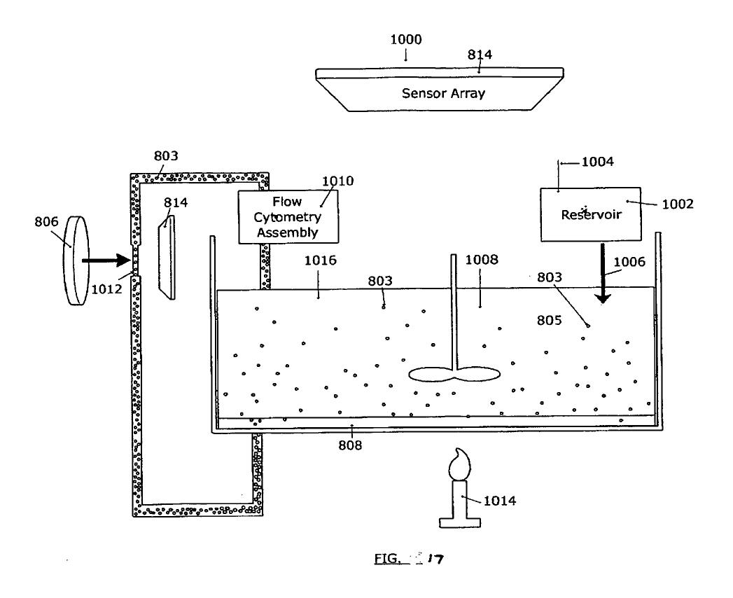

FIG. 15 is a schematic illustration of an assay process 800 that is adapted to determine the kinetic changes in absorbance and/or transmittance and/or optical density and/or light scattering of a particular cell sample. Referring to FIG. 15, and also to FIG. 17, and in the preferred embodiment depicted therein, a medium comprised of the single cells isolated in step 16 of FIG. 1 is preferably fed into a reservoir 1002 by means of line 1004.

In one embodiment, and referring to FIG. 15, a beam of light 804 impacts a cell 803 within a cell medium.

In another embodiment, cell or cells 803 are malignant, and it/they are contacted with light rays 804 emitted by one or more light sources 806 (see FIG. 15). In the embodiment depicted, the cells 803 are disposed in a culture medium 807 which, in turn, is preferably disposed in a culture well 805. As will be apparent, these elements are not drawn to scale to facilitate ease of comprehension.

In the preferred embodiment depicted in FIG. 15, the light rays 804 are preferably emitted substantially perpendicularly to the layer of cells 803. The light source may be one or more of light sources 312 depicted in FIG. 6.

The amount of light emitted by light source 806 is preferably measured by sensor 810, which also determines the amount of light that is transmitted from sensor 810 through to cell(s) 803. Additionally, the sensor 810 measures the amount of light that is reflected back to sensor 810 (see rays 812, 814, and 816).

The sensor 810 may be adapted, e.g., to measure the amount of light scattering. Means for measuring such light scattering are well known to those skilled in the art. Reference may be had, e.g., to U.S. Pat. No. 4,915,501 (device for measuring the light scattering of biological cells), U.S. Pat. No. 4,923,298 (device for measuring the speed of moving light scattering objects), U.S. Pat. No. 4,979,818 (apparatus for measuring movement of light scattering bodies in an object), U.S. Pat. No. 5,057,695 (method of measuring the inside information of substance with the use of light scattering), U.S. Pat. No. 5,113,083 (light scattering measuring apparatus using a photodetector mounted on a rotary stand), U.S. Pat. No. 5,239,185 (method and equipment for measuring absorbance of light scattering materials), U.S. Pat. No. 5,481,113 (method for measuring concentrations of components with light scattering), U.S. Pat. No. 5,712,167 (method of measuring Amadori compound by light scattering), U.S. Pat. No. 5,844,239 (optical measuring apparatus for lights scattering), U.S. Pat. No. 5,870,188 (measuring method by light scattering), U.S. Pat. No. 6,697,652 (fluorescence, reflectance, and light scattering spectroscopy for measuring tissue), U.S. Pat. No. 6,750,967 (light scattering measuring probe), U.S. Pat. No. 6,833,918 (light scattering particle size distribution measuring apparatus), and the like. The entire disclosure of each of these United States patents is hereby incorporated by reference into this specification.

In one preferred embodiment, one may use the measuring devices disclosed in U.S. Pat. No. 4,673,288 (flow cytometry) and U.S. Pat. No. 4,818,103 (flow cytometry), the entire disclosure of which is hereby incorporated by reference into this specification.

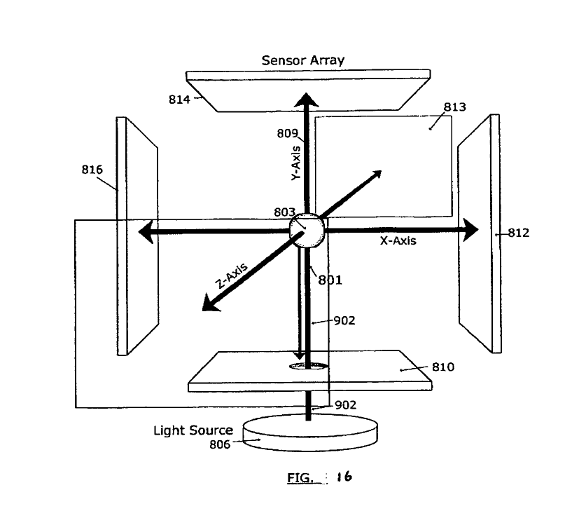

U.S. Pat. No. 4,673,288 discloses and claims (see claim 1) “1. A flow transducer comprising means defining an aperture having an axis, said aperture having at least one flat side, means defining an inlet chamber and an outlet chamber immediately adjacent the aperture along its axis, said inlet and outlet chambers having walls disposed at an angle of at least 5° relative to the plane of the aperture, said inlet and outlet chambers at a distance from the aperture of twice the width of the aperture in a plane through its axis having cross-sectional areas at least 10 times the cross-sectional area of said aperture.” U.S. Pat. No. 4,818,103 discloses and claims (also see claim 1) “1. A flow transducer comprising means defining an aperture having an axis, said aperture having at least one flat side, means defining an inlet chamber and an outlet chamber immediately adjacent the aperture along its axis, at least one of said inlet and outlet chamber having walls disposed at an angle of at least 5° relative to the plane of the aperture, said at least one of said inlet and outlet chambers at a distance from the aperture of twice the width of the aperture in a plane through its axis having a cross-section area at least 10 times the cross-sectional area of said aperture.” In one embodiment, the devices of U.S. Pat. Nos. 4,673,288 and/or 4,818,103 are adapted to make the kinetic measurements described in FIGS. 15 and 16.

Referring again to FIG. 15, and in the preferred embodiment depicted therein, it will be seen that, in addition to sensor array 810, there are also preferably present sensor arrays 812, 814, 816, and others. These sensor arrays are preferably comprised of sensor means for measuring light scattering, optical density, absorbance, transmittance, and other energy-related properties such as, e.g., temperature, pressure, etc. As will be apparent, in the kinetic process depicted in FIG. 15, a series of graphs can be constructed showing the effect of any particular agent upon any one or more of the physical properties of the cell layer and/or its chemical properties and/or its optical properties and/or its biological properties and/or its biochemical and/or any other of its properties.

In one preferred embodiment, hinted at in FIG. 15, a multiplicity of sensors 810/812/814/816 et seq. are disposed circumferentially around the culture chamber 805 in a 360 degree orientation vis-à-vis such chamber 805 such that light emitted from such chamber in any direction or any axis can be captured by one or more of such sensors. This concept is illustrated schematically in FIG. 16.

FIG. 16 illustrates what happens when a quantum of light 801 contacts a cell 803. Some of the light 811 is reflected back directly to the sensor array 810 (see FIG. 5, and also see FIG. 16) The light 811 that is reflected back to the sensor 810 is referred to as back light scatter in this specification. The change in back light scatter over time may be measured by the process of this invention.

Referring again to FIG. 16, a portion of the quantum of light that impinges upon cell 803 is absorbed by such cell 803. By measuring and monitoring the total amount of light that impacts cell 803 and deducting the amount of light that is either transmitted and/or scattered, one can continually determine the amount of light 801 that is absorbed. The change in absorbance over time may be measured by the process of this invention.

Referring again to FIG. 16, a portion 809 of the quantum of light that impinges upon cell 803 is transmitted through said cell is a direction that is substantially parallel to the incoming quantum of light 801. One thus can continually monitor the amount of light that is transmitted by cell 803, and the change in transmittance over time may be measured by the process of this invention.

Referring again to FIG. 16, a certain amount of the light 801 that impacts cell 803 will be side scattered in the “x-axis) substantially perpendicularly to the direction of the incoming light 801. The change in side scattering over time may be measured by the process of this invention.

Similarly, one may measure the amount of light scattered in the z axis, which light will be perpendicular to the light in the x-axis and/or the y-axis. Many other different parameters also may be measured by specifying, e.g., a particular point in the x,y,z coordinate system and determining how the light at that point varies in time.

The processes depicted in FIGS. 15 and 16 measure a sample of cells that are viable and, thus, may be changing their properties. In the embodiment depicted in FIG. 17, a process is provided for measuring these same cells when other parameters are varied, such as, e.g., their concentrations.

Referring again to FIG. 15, and in the preferred embodiment depicted therein, the cell chamber 805 is preferably comprised of an agent, such as a chemotherapeutic agent, a hormone, an infectious agent, etc., that may affect the viability of the cell 803. However, the system depicted in FIG. 15 is somewhat static in that the concentration of such agent, and/or the concentration of the cell 803, often does not vary very much.

In life, however, the situation is often much more dynamic. An agent that is added to a biological system changes its concentration as it contacts bodily tissue, and the bodily tissue, especially if it is mobile, also often changes its concentration. Thus, the process depicted in FIG. 17 allows one to monitor the kinetic changes in a system over time as one or more of such concentration and/or other properties are varied.

FIG. 17 illustrates a continuous assay system 1000 that is adapted to determine the changes in a dynamic system, in the embodiment depicted, one or more cell viability agents (such as, e.g., cytotoxic agents like paclitaxel) may be added to reservoir 1002 via line 1004, and one or more of the material in reservoir 804 may be added to chamber 805 (see FIG. 15) via line 1006. In a living biological system, the concentration of, e.g., cytotoxic agents is not necessarily static, and the device of FIG. 17 allows you to test the effects of changes in such agents.

Similarly, in a living system, the cells 803 are not necessarily quiescent. The use of a mixer 1008 allows one to stir such cells 803.

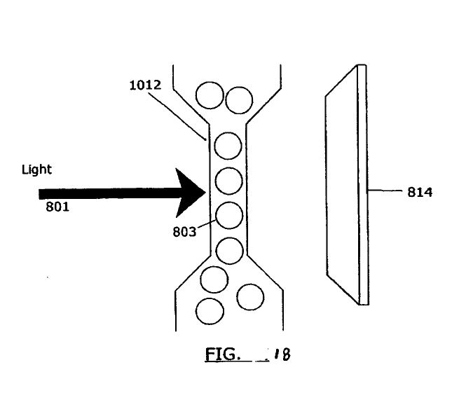

The use of a flow cytometer assembly 1010 allows one to continually move a portion of the cells in the chamber 805 past a single cell inspection station described in greater detail by reference to FIG. 18.

The use of a Bunsen burner, 1014, allows one to change the temperature conditions the cell 803 is subjected to. Similarly, gas can be bubbled into the system via line 1016 to vary the oxygen content of the system.

In the preferred embodiment depicted in FIG. 18, the cells 803 are preferably contacted with light quanta 801, and the responses of such cells 803, in the x and/or y and/or y directions, or at any point in the x, y, z coordinate system, is then determined. In one aspect of this embodiment, it is preferred to contact light 801 with a collection of single cells 803, at point 1012.

Another Preferred Embodiment

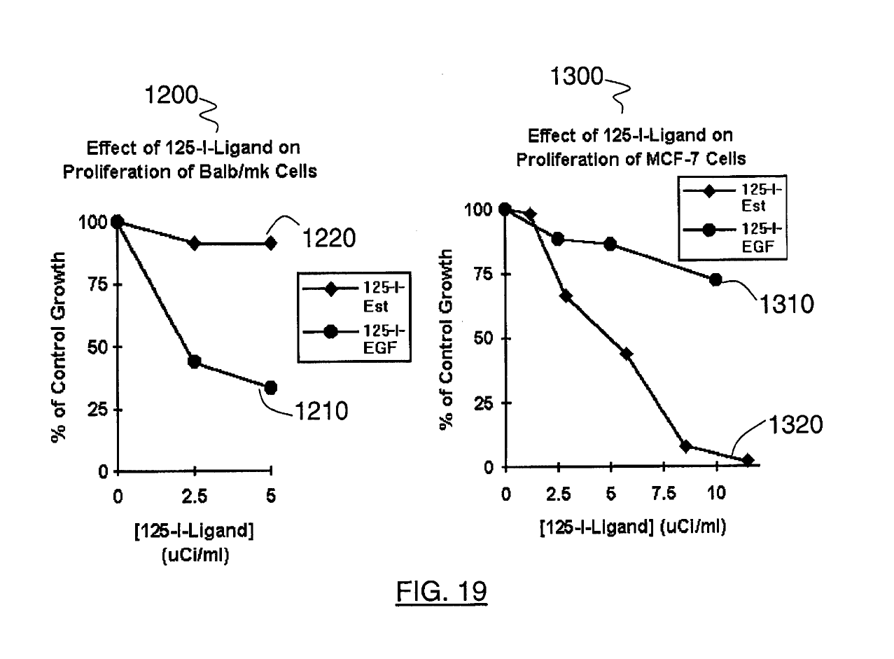

FIG. 19 is a representation of the results of exposing cells to various lineage specific hormones. In one particular embodiment, see chart 1200, epithelial carcinoma cells, 1210, and ovarian cancer cells, 1220, were exposed to radiolabeled epithelial growth factor (EGF). As known to those skilled in the art, a radiolabeled ligand can be used to kill cells that possess receptors for the particular ligand. Reference may be had to U.S. Pat. No. 6,565,827 (radioimmunotherapy of lymphoma using anti-CD20 antibodies), U.S. Pat. No. 6,287,537 (radioimmunotherapy of lymphoma using anti-CD20 antibodies), U.S. Pat. No. 6,015,542 (radioimmunotherapy of lymphoma using anti-CD20 antibodies), and U.S. Pat. No. 5,843,398 (radioimmunotherapy of lymphoma using anti-CD 20 antibodies). The entire disclosure of these United States patents are hereby incorporated by reference into this specification. As is readily apparent, more than 50 percent of the epithelial carcinoma cells are killed upon exposure to the radiolabeled epithelial growth factor but less than 10 percent of the ovarian cancer cells are killed upon exposure.

In another embodiment (see graph 1300) epithelial cells, 1310, and ovarian cancer cells, 1320, are exposed to radiolabeled estrogen. Over 90 percent of the ovarian cancer cells are killed upon exposure to the radiolabeled estrogen but less than 30 percent of the epithelial carcinoma cells are killed upon exposure.

Another Preferred Embodiment

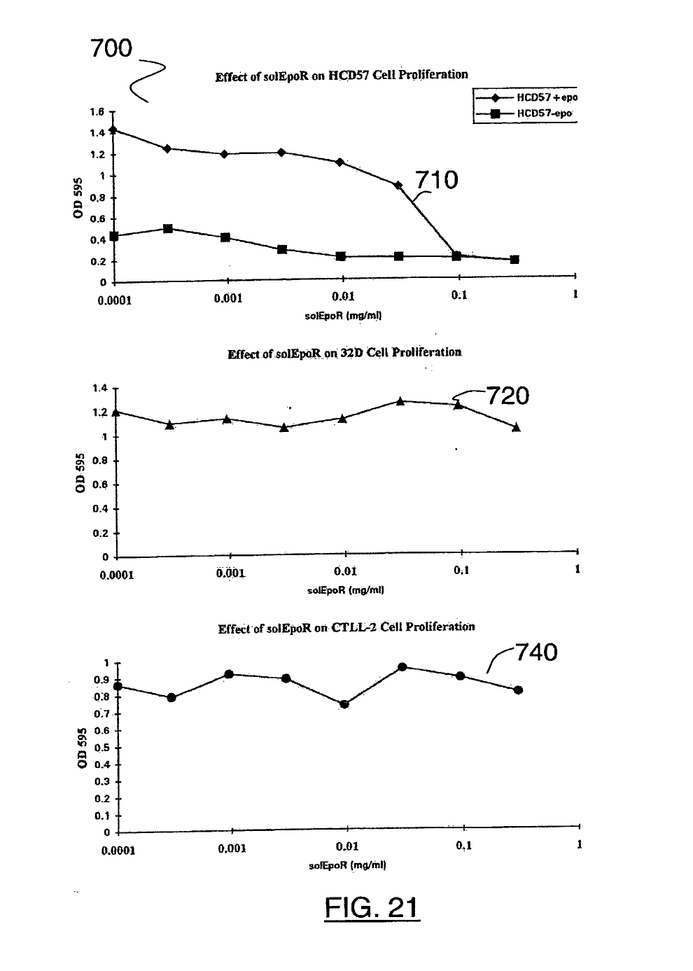

FIG. 21 is a representation of the results of exposing cells to various lineage specific hormone inhibitors 700. In one particular embodiment, depicted in graph 710, a soluble receptor, which will bind free hormone, e.g. erythropoietin, is added to cell cultures of erythroleukemia cells and brings about the death of these cells by depriving them of their essential viability hormone. In this embodiment, adding the soluble receptor which binds erythropoietin does not induce the death of cells of other specific cell lineages, e.g. myeloid cancer cells, 720, and lymphoid cancer cells, 740, and the like.

A Process for Confirming the Existence of Mesenchymal Chrondrosarcoma by the Over Expression of Fibroblast Growth Factor Receptor Like 1 Gene

In this portion of the specification, applicant will discuss a process for detecting the existence of Mesenchymal Chrondrosarcoma comprising the steps of analyzing tumor cells and determining the extent to which such tumor cells contain fibroblast growth factor receptor-like 1 protein. In one embodiment, when such protein is expressed at least 1,000 percent more than in non-cancerous cells, such overexpression is an indicium of the existence of the Mesenchymal Chrondrosarcoma cancer.

Mesenchymal Chrondrosarcoma has been discussed in the literature. Reference may be had, e.g., to an article by Kristin Baird et al., “Gene Expression Profiling of Human Sarcomas: Insights into Sarcoma Biology” (Cancer Res. 2005; 65: [20]. Oct. 15, 2005). Excerpts from this article are presented below.

“Sarcomas are a biologically complex group of tumors of mesenchymal origin. By using gene expression microarray analysis, we aimed to find clues into the cellular differentiation and oncogenic pathways active in these tumors as well as potential biomarkers and therapeutic targets. We examined 181 tumors representing 16 classes of human bone and soft tissue sarcomas on a 12,601-feature cDNA microarray. Remarkably, 2,766 probes differentially expressed across this sample set clearly delineated the various tumor classes. Several genes of potential biological and therapeutic interest were associated with each sarcoma type, including specific tyrosine kinases, transcription factors, and homeobox genes. We also identified subgroups of tumors within the liposarcomas, leiomyosarcomas, and malignant fibrous histiocytomas. We found significant gene ontology correlates for each tumor group and identified similarity to normal tissues by Gene Set Enrichment Analysis. Mutation analysis done on 275 tumor samples revealed that the high expression of epidermal growth factor receptor (EGFR) in certain tumors was not associated with gene mutations. Finally, to further the investigation of human sarcoma biology, we have created an online, publicly available, searchable database housing the data from the gene expression profiles of these tumors (http://watson.nhgri.nih.gov/sarcoma), allowing the user to interactively explore this data set in depth. (Cancer Res 2005; 65(20): 9226-35).”

“Sarcomas are malignant tumors of mesenchymal origin with 15,000 soft tissue and bone sarcomas newly diagnosed in the United States annually. Although sarcomas represent only 1% of all human malignancies, they have distinctive biological characteristics, which include a high incidence of aggressive local behavior and a predilection for metastasis. Several sarcomas such as Ewing's sarcoma, synovial sarcoma, alveolar rhabdomyosarcoma, and myxoid liposarcoma tend to occur in younger patients and are characterized by tumor-specific chromosomal translocations. In contrast, other sarcomas such as leiomyosarcoma and malignant fibrous histiocytoma lack specific translocations and have a chaotic karyotype accompanied by frequent chromosome copy number changes. This latter group occurs more frequently in older adults and includes several types of sarcomas that lack known disease-specific chromosome translocations or mutations, but may contain mutations in RB1, CDKN2A, and TP53. Investigation of these and other aspects of sarcoma biology have provided insights into broadly relevant fundamental mechanisms of oncogenesis (for review, see ref. 1) and may have profound implications in the development of therapeutic intervention. This is exemplified by the successful treatment of gastrointestinal stromal tumors, which frequently have activating mutations of KIT, with the tyrosine kinase inhibitor, imatinib mesylate. Although genetic alterations, particularly fusion genes arising from translocations, have been identified in many sarcomas, the function of the fusion gene products are not well understood and their downstream targets have not been fully identified (1). Additionally, in tumors that lack specific chromosomal translocations, there may well be additional oncogenic mutations to be described. Furthermore, “second hits” or additional genetic mutations, thought to be essential in cancer development, have rarely been identified in sarcomas (1).”

“Progress in the evaluation of sarcomas has been limited not only by their rarity, but also by their histologic diversity and genetic complexity, making high-throughput tumor profiling a critical tool to advance understanding of sarcoma tumor biology. Studies of human sarcoma samples using microarray technology first began with a report on seven alveolar rhabdomyosarcoma on a 1,238-feature microarray (2). Since then, there have been remarkable advances in microarray technology leading to a growing body of gene expression studies focused on characterizing this complex group of tumors (3-5). Recent studies have described gene signatures associated with poor clinical outcome in leiomyosarcoma (6) and Ewing's sarcoma (7), diagnostic classification (8), and novel biomarkers in dermatofibrosarcoma protuberans (9) and clear cell sarcoma (10).”

“Previous studies have generally focused on a limited number of histotypes and have typically separated bone and soft tissue sarcomas. We sought to generate a technically uniform gene expression data set that would allow a broad view of the more frequent bone and soft tissue sarcoma types. In this study, we utilized gene expression array analysis and denaturing high-performance liquid chromatography and immunohistochemistry on tissue microarray to evaluate the largest set of human sarcoma samples studied by high-throughput genetic techniques to date. Gene expression data was processed by integrating standard cluster analysis methods with more recently developed approaches, such as gene ontology analysis and gene set enrichment analysis. Our primary goal was to present an in depth evaluation of the expression profiles found in human sarcomas which could be used as a basis for the identification of their key biological features. To achieve this goal, we have established tumor specific profiles with highly significant gene lists, created gene ontology profiles, identified expression of potentially critical genes and pathways and developed a searchable database which makes these data available to the sarcoma community. Through this comprehensive approach, we gained additional insight into the genetic diversity and complexity of sarcomas, including clues regarding their origin, differentiation and pathophysiology.

The Fibroblast Growth Factor Receptor-Like 1 Protein

In one process of the present invention, the relative expression of a protein identified as “FGFRL1” is determined. As is known to those skilled in the art, FGFRL1 is a novel member of the FGF receptor family. Reference may be had to, e.g., (1) C. Schild et al., “Aberrant expression of FGFRL1, a novel FGF receptor, in ovarian tumors” (Int J Mol. Med. 2005 Dec; 16[6]:1169-73; (2) Expression of FGFRL1, a novel fibroblast growth factor receptor, during embryonic development ([Int J. Mol. Med. 2006] PMID: 16525717); and (3) Characterization of FGFRL1, a novel fibroblast growth factor (FGF) receptor preferentially expressed in skeletal tissues, ([j. Biol Chem. 2003] PMID: 12813049).

The FGFRL1 gene, and the protein expressed by it, is also described in International patent publication WO02057312A2, the entire disclosure of which is hereby incorporated by reference into this specification. As is disclosed in this published patent application, “The expression of the novel FGFR-like gene was examined on a Northern blot containing RNA from 12 different human tissues (brain, heart, skeletal muscle, colon, thymus, spleen, kidney, liver, small intestine, placenta, lung, peripheral blood leukocytes; Clontech Laboratories). No expression was observed in any of these tissues even after prolonged exposure time (not shown). Since the commercial blot did not contain any cartilaginous tissues, a fresh Northern blot with RNA from fetal human tissues was prepared. In this case, the probe gave a strong signal with RNA from vertebrae, bone and total embryo, which at this developmental stage still contain a large proportion of cartilage (FIG. 1C). A very faint signal was also obtained with RNA from fetal muscle. The migration position of the mRNA corresponded to a size of 3.4 kb which is consistent with the length of the cDNA sequence, assuming a poly(A) tail of 300 nucleotides”

In the abstract of the Schild article (“Aberrant expression of FGFRL1, a novel FGF receptor, in ovarian tumors”), the author disclosed that: “FGFRL1 is a novel member of the FGF receptor family. It is expressed at very low levels in a great variety of cell lines and at relatively high levels in SW1353 chondrosarcoma cells, MG63 osteosacroma cells, and A204 rhabdomysarcoma cells. Screening of 241 different human tumors with the help of a cancer profiling array suggested major alterations in the relative expression of FGFRL1 in ovarian tumors. Several tumors were found to exhibit a significant decrease in the expression of FGFRL1 in the tumor tissue relative to the matched control tissue. One ovarian tumor showed a 25-fold increase in the relative expression. Since FGFRL1 appears to be involved in the control of cell proliferation and differentiation, its aberrant expression might contribute to the development and progression of ovarian tumors.”

Applicant has discovered that FGFRL1 protein is aberrantly expressed in Mesenchymal Chrondrosacroma and, generally, is is expressed from at least 1,000 to 1,000,000 percent as much as it is expressed in normal tissue. This finding is utilized in applicant's process with the use of the FGFRL1 protein as a biomarker for Mesenchymal Chrondrosacroma.

The expression of the FGFRL1 gene may be measured by conventional means such as, e.g., gene expression profiling. Gene expression profiling is well known to those skilled in the art and is referred to in the claims and specifications of U.S. Pat. No. 6,203,988 (DNA fragment preparation method for gene expression profiling) as well as published United States patent applications 20030232364 (Diagnosis, prognosis and identification of potential therapeutic targets of multiple myeloma based on gene expression profiling), 20040009523 (Diagnosis, prognosis and identification of potential therapeutic targets of multiple myeloma based on gene expression profiling), 20040339245 (Methods and algorithms for performing quality control during gene expression profiling on DNA microarray technology), and 20050112630 (Diagnosis, prognosis and identification of potential therapeutic targets of multiple myeloma based on gene expression profiling). The entire disclosure of each of these United States patents is hereby incorporated by reference into this specification.

Instead of measuring the RNA transcripts of the FGFRL1 gene, one may alternatively or additionally measure the relative expression of the FGFRL1 protein. This may be done by conventional means. Reference may be had, e.g., to an article by S. Varambally et al. entitled “Integrative genomic and proteomic analysis of prostate cancer reveals signatures of metastatic progression” (Cancer Cell. 2005 Nov; 8(5):393-406). In the abstract of this article, it is disclosed that: “Molecular profiling of cancer at the transcript level has become routine. Large-scale analysis of proteomic alterations during cancer progression has been a more daunting task. Here, we employed high-throughput imunoblotting in order to interrogate tissue extracts derived from prostate cancer. We identified 64 proteins that were altered in prostate cancer relative to benign prostate and 156 additional proteins that were altered in metastatic disease.”

Applicant has analyzed the data presented in the Varambally et al. article and discovered that the FGFRL1 protein was aberrantly expressed only with metatstatic prostate cancer and not with the non-metastatic variety or with benign prostate. In any event, one may use the immunoblotting technique described in this article to determine the relative concentrations of the FGFRL1 protein in applicant's process, where one is attempting to determine the existence of Menenchymal Chrondroscarcoma.

The use of immunobloblotting to determine the relative concentrations of a protein are well known. Reference may be had, e.g., to U.S. Pat. No. 5,580,780 (Vascular adhesion protein. . . and VAP-1 specific antibodies), U.S. Pat. No. 5,989,815 (Methods for detecting predisposition to cancer at the MTS gene), U.S. Pat. No. 6,946,256 (cell regulatory genes, encoded products, and uses related thereto), and the like. The entire disclosure of each of these United States patents is hereby incorporated by reference into this specification.

Description of Certain Experimental Work Conducted by the Applicant on Live Tumor Cells Derived from Individual Mesenchymal Chondrosarcoma Patients.

The research conducted by the applicant has identified the critical need for obtaining and propagating live cells from human tumors in order to optimally conduct various biological, molecular and pathology analyses which, in turn, are designed to improve diagnosis and treatment of cancer patients. Therefore, the goal was to use these live tumor cells to gain patient specific tumor information that would improve the chances of a cancer patient's clinical team coming up with a successful treatment of this rare mesenchymal chondrosarcoma. During the project, we were able to perform, as rapidly as tissue availability permitted, various morphological, molecular, developmental biological, endocrine and drug response analyses relating to a specific variant of chondrosarcoma, known as Mesenchymal Chrondrosarcoma (MC). Through specially designed procedures, there was acquired, grown in tissue culture, and analyzed fresh live tumor tissues from several mesenchymal chondrosarcoma patients.

Elsewhere in this specification, there is described a novel bioinformatics approach to therapy selection and discovery—an artificial intelligence, medical mapping system, called Hamms. The Hamms approach is based on the hypothesis that integration of sufficient genomic and biological response data to precisely define the maturation status of the tumor clone (i.e. specific receptors, surface antigens, kinases, etc.) within a particular pathway of differentiation, (e.g. blood, or in MC's case, cartilage) will yield valuable diagnostic and therapeutic information. This information provides clues with which teams can a) deduce the best therapy among current options, b) allow new combination therapies to be considered, and c) discover or design new, lineage specific drugs that should be more effective with less widespread side-effects. The live cells, propagated in culture or mice, provide the test beds for these ideas.

One of the objectives of this method was to allow clinical investigators to relate the therapeutic response of individual tumors to their degree of mesenchymal chondorcyte differentiation—as evidenced by lineage and stage specific, gene expression patterns, with a particular emphasis on the expression of viability hormone receptors. Specifically, the method is directed toward: (a) collecting live tissue and processing the malignant cells, (b) defining the growth factor/viability hormone receptor status by live cell gene expression analysis using combinatorial PCR analysis of Mesenchymal Chondrosarcoma cells, (c) generation of disease-specific and patient specific tumor models for personalized research and drug discovery, (d) defining the response of said tumor cells to potential therapeutic agents, and (e) defining new diagnostic and prognostic biomarkers and novel gene or protein targets that will foster discovery of new therapeutic options for these patients.

A. Collecting Live Surgical Tissue and Processing the Malignant Cells

When a patient's sample is received for analysis it is processed to make a single-cell suspension of malignant cells. The mesenchymal chondrosarcoma samples are manually minced into particles of 2 to 3 mm greatest dimension, suspended in RPMI-1640 medium containing growth factors and hormone specific for that tissue. Fibroblast Growth Factors and other viability hormones are added when appropriate.

In one embodiment, the minced tumor tissue is pressed sequentially through sterile 60 and 45 μm nylon cell strainers and pipetted gently and repeatedly to make a single-cell suspension.

An aliquot of these single-cell suspensions is analyzed for viability and purity of the malignant cells. The viability is preferably evaluated by exclusion of the fluorescent dye 7-amino actinomycin D (7-AAD). The purity is assessed by comparison with the flow cytometric phenotyping performed on either a previously obtained sample of the patient's tumor or on a portion of the same sample submitted for diagnostic evaluation. The results of the flow cytometry analysis of the preliminary single-cell suspension indicate a sample of high purity.

The resulting tumor cell suspensions are suitable for either direct use in for gene expression analyses, or to generate cell lines either in cell culture or through passage as xenografts in immunocompromised mice.

B. Defining the Growth Factor/Viability Hormone Receptor Status by Live Cell Gene Expression Analysis using Combinatorial PCR Analysis of Mesenchymal Chondrosarcoma Cells

Open-architecture molecular libraries (RAGE) prepared from fresh tissue rather that from frozen or paraffin-embedded tissue.

RAGE libraries were prepared and analyzed from individual MC patient tumors, cell lines and xenographs to: a) indicate the factors needed to create MC cell lines, b) identify MC-specific hormone receptors/ligands, cell surface or intracellular biomarkers as potential anti-cancer drug targets, and c) provide alternative molecular materials for confirmation, extension, and comparison of other molecular studies on MC and other MC tumor cells. The approximately 300 genes selected for testing were based on my observations and analyses of our data, literature searches, as well as the data compiled by other investigators involved in the MC research.

The results from our RAGE analysis confirmed the value of this strategy, in that it allowed us to identify a number of specific genes associated with the mesenchymal chondrosarcoma lineages and notably expressed in MC -1 cells. Thus, our hypothesis was supported that defining the molecular lineage phenotype would lead directly to practical and clinically relevant information. First, the new growth factor receptor (e.g. FGFRLI, etc) information allowed us to better expand the MC -1 cells for drug testing and differentiation-induction studies. Second, the gene expression data permitted the prediction of effective therapies among current drugs (e.g. Sutent which targets FGFR and Avastin which targets VEGF). Third, multiple new genes were identified in MC -1 cells which could serve as new therapeutic targets for antibody or small molecule drug development—including not only the surface receptor, FGFRL1 but also a previously unidentified gene that was expressed in MC -1 cells. It should also be noted that on each tissue specimen obtained from MC, or other MC patients, expanded open-architecture molecular libraries (cDNA, RNA for RAGE, microarray studies) were prepared for distribution and analysis by other collaborators on the MC team.

In one embodiment of the invention of this specification, there is disclosed the fact that FGFRL1 can serve as diagnostic and therapeutic biomarker as well as an excellent gene target for which to develop new drugs that will target cells expressing the FGFRL1 cell surface protein.

The significance of the MC investigations is manifold. MC is an extremely rare sarcoma. There is little information concerning the cell biology, molecular biology or therapeutic response. We have discovered a clear association of the Fibroblast Growth Factor-L1 in and only in Mesenchymal Chondrosarcoma cells—qualifying this receptor protein as a biomarker for MC. The availability of multiple primary cultures is an unprecedented resource. Further study of these cultures could yield important new information relevant to identification of therapeutic targets and would enable testing of currently approved kinase inhibitors, metabolic inhibitors, apoptosis inducers or genotoxic agents singly or in combinations. In particular, the potential for therapeutic differentiation of MC needs to be explored. For example the multilineage potential could be investigated by addition of some appropriate growth factors and by comparison of gene expression profiles. Since MC is naturally slow growing, it is conceivable that in vivo therapeutic differentiation as a means to prolong disease remission might become feasible. In principle, this would be comparable to treatment of M3 acute leukemia with retinoic acid. Extended gene mapping/expression studies also should lead to an improved understanding of key signal transduction pathways critical to survival/growth of MC. The potential for testing of protein kinase inhibitors, interfering RNA or antisense oligonucleotides might enable identification of specific therapeutic targets and guide a more rational application or development of clinically acceptable kinase inhibitors which could be effective alone (such as Gleevec) or combined with differentiation therapies or conventional chemotherapeutics. Finally, the new genes and novel receptor transcript variants, discovered in MC -1 cells and confirmed in other MC tumors, offer potential new therapeutic targets and could lead to a better understanding of the differentiation of sarcoma tumors and their untransformed counterparts.

The FGFR-L1 protein association with MC was discovered because we have uniquely employed three innovations not used before (A) fresh live tumor tissue taken sterile directly from the surgical suite and maintained alive via a number of specialized cocktails and procedures, and (B) analysed for hormone receptor gene expression using the RAGE (Rapid Analysis of Global Expression) Combinatorial PCR technology systems (U.S. Pat. Nos. 6,221,600 and 7,115,370) and (C) Using this hormone receptor knowledge, we generated two types of fresh tissue cell lines—personalized to each patient from which the tumors were derived. One type of cell line was generated in vitro, i.e. in tissue culture and a second type, Xenograft cell lines, was generated in vivo by inoculating the live tumor cells into immunocompromised mice. These fresh tissue cell lines, and the RAGE molecular libraries derived from the lines, 1permitted us a unique opportunity. to prove the association of FGFRL1 with Mesenchymal Chondrosarcoma cells. These in vitro and in vivo cell lines also provide a biological model in which to discover, test and study mechanisms of candidate drugs which target the FGFRL1 protein or other drug targets which may be preferentially expressed in this class of tumor cells.

Methods Used for Open Architecture Libraries (RAGE Studies)

Use of combinatorial oligonucleotide polymerase chain reaction to perform live tissue gene expression profiling.

In this section of the specification, there is described a method which allows for the determination of changes in “live cell” gene expression related to individual Mesenchymal Chondrosarcoma patient tumor characteristics as well as the response of each tumor population, or sub-population, to potential therapeutic agents.

The degree of differentiation or physiological state of a cell, a tissue or an organism is characterized by a specific expression status, i.e., the degree of transcriptional activation of all genes or particular groups of genes. The molecular basis for numerous biological processes that result in a change in this state is the coordinated transcriptional activation or inactivation of particular genes or groups of genes in a cell, an organ or an organism. Characterization of this expression status is of key importance for answering many biological questions. Changes in gene expression in response to a stimulus, a developmental stage, a pathological state or a physiological state are important in determining the nature and mechanism of the change and in finding cures that could reverse a pathological condition. Patterns of gene expression are also expected to be useful in the diagnosis of pathological conditions, and for example, may provide a basis for the subclassification of functionally different subtypes of cancerous conditions.

The object of the present study is to provide a method for gene expression analysis which exceeds the capabilities of the state of the art. Thus, the present invention described herein provides novel improvements to the art of gene expression analysis, particularly using combinatorial oligonucleotide polymerase chain reaction with labeled linkers and amplification of restriction fragments comprising nonidentical ends.

The method we used (see U.S. Pat. No. 7,115,370. the entire disclosure of which is hereby incorporated by reference into this specification) allows for the determination of changes in gene expression in multiple genes, known and unknown, in a rapid, quantitative and cost-effective fashion. This invention improves on the combinatorial oligonucleotide polymerase chain reaction technology, particularly which is described in U.S. Pat. No. 6,221,600 (the entire disclosure of which is hereby incorporated by reference into this specification), which is used to determine the differential expression of mRNA from cells or tissue. We have use these methods for detecting the frequency distribution of all polyadenylated mRNAs in Mesenchymal Chondrosarcoma samples at any selected time or condition. The method in use reduces the complexity of analysis by ensuring that only a single unique fragment is derived from each molecular species of polyadenylated mRNA. Either the entire genome or a subset can be analyzed, and a single set of reagents and reaction conditions is sufficient for analysis of the complete genome. The technique allows for multiple samples to be analyzed simultaneously. The results generated from this method in use are quantitative and proportional to the level of expression of the particular gene.

A unique feature of this method that distinguishes it from all DDRT methods is that a one-to-one correspondence exists between each molecular species of polyadenylated RNA and a PCR product of a particular length derived with a particular pair of PCR primers. Knowledge of a gene sequence therefore can be used to select the correct pair of primers to use for amplification and to predict the length of the corresponding product. This feature is also advantageous when combinatorially surveying the entire (genome) transcriptome. The length of the amplimer products, along with the information on the primers can be plugged into the database to identify the differentially expressed genes.

The present method in use improves on combinatorial oligonucleotide polymerase chain reaction technology by facilitating the recovery of only one unique restriction fragment of each cDNA species in a collection of products. The method in use utilizes an anchorable moiety to eliminate the recovery of rare restriction fragments with two identical ends that result upon restriction digestion with the second restriction enzyme. Failure to remove fragments with two identical ends would result in undesirable background upon subsequent amplification steps. Only the fragments comprising two nonidentical ends are isolated from other fragments via an anchorable linker, such as a biotinylated linker. This improvement dramatically improves the signal to noise ratio by eliminating amplification of templates that only contain identical ends. Use of the anchorable linker also facilitates improved recovery of the desired restriction fragments through specific, high affinity binding of biotin to streptavidin. The present method in use also is directed to the compositions generated by the methods described herein. In a specific embodiment, a composition is a linker-ligated fragment from a DNA, such as a cDNA, referred to as a RAGEtag.

One embodiment of the method in use involves a method comprising obtaining DNA molecules, which includes an anchorable moiety, and cleaving the DNA molecules with a first restriction endonuclease. The immobilized fragments are then digested with a second restriction endonuclease, cleaving the fragment from the anchor. The released fragments are then precipitated with carboxyl-magnetic beads and released from the beads. On occasion more than one restriction fragment from a single molecule of DNA may result from the second restriction digestion. Only the restriction fragments with two non-identical ends are desired. Two distinct linkers are then ligated to the non-identical cut ends of the DNA fragments. The linker that attaches to the fragments generated from the first restriction enzyme digestion has an anchorable moiety. After ligation of the linkers the desired fragments containing non-identical ends are isolated by immobilizing those fragments on the anchor via the anchorable moiety. The fragment library is then amplified. The order of the restriction digests may be reversed, thereby representing a more complete share of the DNA present in the sample. When the order of the restriction enzymes is reversed, the linker that has the anchorable moiety should also be switched.

In a further embodiment of the method in use, mRNA is reverse transcribed to cDNA with an oligo-dT primer. It is further envisioned that reverse transcription may also be initiated at a random hexamer. The oligo-dT primer was attached to a ligand, for example biotin or an antibody. Where the oligo-dT includes a ligand, this ligand is the means through which the cDNA is immobilized to a substrate. Where the ligand is biotin, the biotin was attached to streptavidin.

In another embodiment, the initial immobilization of the DNA may take place subsequent to the initial restriction digestion. The immobilization will occur at the anchorable moiety via a means of adhering. The means of adhering may facilitate either a covalent or non-covalent interaction. The anchorable moiety was located at either the 5′ or 3′ end of the DNA. The means of adhering was either biotin or an antibody.

In a preferred embodiment of the method in use, at least one linker is attached to one end of a fragment from the cDNA, and preferably linkers will attach to both ends of the fragment. In specific embodiments, the linker oligonucleotides will adhere to the cut end of the DNA fragment via ligation or attachment.

In an embodiment of the present method in use, a linker-ligated fragment is anchored. In a specific embodiment, the means of anchoring is via an anchorable moiety incorporated into one of the linkers. The means of anchoring may comprise either a covalent or non-covalent interaction. A skilled artisan recognizes the anchorable moiety could be at or near the 5′ or 3′ end of the linker.

In specific embodiments, the anchorable moiety is a ligand. Examples include biotin or an antibody. Where the anchorable moiety comprises a ligand, this ligand is the means through which the DNA is immobilized to a substrate. Where the ligand is biotin, the biotin was attached to streptavidin.

In another embodiment of the method in use, the amplification of the fragment is initiated at primers of a sequence complementary to the first and second linkers respectively. It is further envisioned that this amplification reaction may include: a first amplification primer in which the 5′ sequence of the primer is complementary to the first linker sequence and the 3′ sequence comprises a specificity region; a second amplification primer, wherein the 5′ sequence of said primer is complementary to said second linker sequence and the 3, sequence comprises a specificity region. This method was further modified to consist of an array of combinations of alternate amplification primers such that the specificity region facilitates the amplification of a substantial percentage of the different sequence templates within a sample. Such an array was simplified by carrying it out in a multi-well plate.

Amplification of the samples was further enhanced by pre-amplification with primer pairs complementary to the first and second linker sequences, respectively, prior to amplification with said amplification primers. Further, a partial nucleotide sequence identification of the amplified products was facilitated by the sequence of the primers used for the amplification. such identification was carried out with the aid of a computer program. It is further envisioned that the identification of the amplified DNA was based on length.

The 3, specificity region of the first and second primers was 3 nucleotides long. It is further envisioned that such 3′ regions was either 4, 5, 6, 7 or even 8 base pairs long.

Amplification of the fragments may occur through either the polymerase chain reaction, nucleic acid sequence based amplification, transcription mediated amplification, strand displacement amplification, ligase chain reaction or any other method recognized by a person of ordinary skill in the art to be useful in the amplification of nucleic acid.

The one or both of the restriction enzymes used to digest the immobilized DNA molecule have either a four, five, six, seven or eight base recognition site. In a preferred embodiment of the method in use, the one or both of the restriction enzymes will have a four base pair recognition site. Such restriction enzymes might include but is not limited to: NlaIII, DpnII, Sau3AI, Hsp92II, MboI, NdeII, Bsp1431, Tsp509 I, Hhal, HinP1I, HpaII, MspI, Taqalphal, MaeII or K2091.

Thus, the procedures of the present method in use comprises a) obtaining a DNA; b) cleaving the DNA with a first restriction endonuclease; c) cleaving the DNA with a second restriction endonuclease, wherein the cleaving results in releasing a fragment having two nonidentical ends from the DNA; d) ligating a first labeled linker to a first end of the fragment; and e) ligating a second linker to a second end of the fragment, wherein the linkage of both linkers to the fragment produces a linker-ligated fragment. In a specific embodiment, the method further comprises the step of obtaining the linker-ligated fragment by the label. In another specific embodiment, the DNA is immobilized. In a further specific embodiment, step b) further comprises removal of fragments cleaved from the immobilized DNA. In an additional specific embodiment, the obtaining step is further defined as isolating the linker-ligated fragment. In another specific embodiment, isolating the linker-ligated fragment is defined as binding the labeled linker-ligated fragment to a bead. In a further specific embodiment, the binding of the linker-ligated fragment to the bead is through the label. In another specific embodiment, the label is biotin and wherein the bead is coated with streptavidin. In a particular specific embodiment, DNA is immobilized on a magnetic bead. In another specific embodiment, the DNA is immobilized on a magnetic bead through a biotin label, wherein the bead further comprises a coating of streptavidin. In a particular specific embodiment, the ligating steps occur concomitantly.

In a specific embodiment, the method further comprises amplification of the linker-ligated fragment. In a specific embodiment, the amplification is by polymerase chain reaction with two different primers. In another specific embodiment, the DNA is non-genomic DNA. In a further specific embodiment, the DNA is cDNA. In an additional specific embodiment, the immobilizing step further comprises a means of adhering. In another specific embodiment, the means of adhering comprises a means of establishing a non-covalent interaction. In another specific embodiment, the means of adhering comprises a means of establishing a covalent interaction. In a further specific embodiment, the means of adhering comprises a ligand. In an additional specific embodiment, the means of adhering is biotin. In an additional specific embodiment, the means of adhering comprises an antibody. In a specific embodiment, the DNA is immobilized at the 3′ end. In a further specific embodiment, the cDNA is reverse transcribed from messenger RNA. In a particular specific embodiment, the reverse transcription is initiated at an oligo dT. In another specific embodiment, the reverse transcription is initiated at a random hexamer. In an additional specific embodiment, the oligo dT is biotinylated. In another specific embodiment, the cDNA is immobilized on a substrate by means of the biotinylated oligo dT. In a specific embodiment, the substrate is streptavidin. In a specific embodiment, the order of the first and the second restriction endonuclease is reversed. In an additional specific embodiment, the amplification is initiated at primers comprising a sequence complementary to the first and the second linkers respectively.

In a further specific embodiment, the amplification is carried out with a primer set comprising a) a first amplification primer, wherein the 5′ sequence of the primer is complementary to the first linker sequence and the 3′ sequence comprises a specificity region; b) a second amplification primer, wherein the 5′ sequence of the primer is complementary to the second linker sequence and the 3′ sequence comprises a specificity region. In a specific embodiment, the DNA fragment is preamplified. In a further specific embodiment, the amplification is performed with an array of combinations of alternate amplification primers. In an additional specific embodiment, the method further comprises identifying the amplified DNA. In a specific embodiment, the identification is based upon length. In another specific embodiment, the identification is performed by a computer program. In a further specific embodiment, the amplification is performed in a multi-well plate. In another specific embodiment, the specificity region of the first amplification primer is 3, 4, 5, 6, 7 or 8 base pairs long. In an additional specific embodiment, the specificity region of the second amplification primer is 3, 4, 5, 6, 7 or 8 base pairs long. In an additional specific embodiment, the amplification comprises polymerase chain reaction, nucleic acid sequence based amplification, transcription mediated amplification, strand displacement amplification or ligase chain reaction. In a further specific embodiment, the first restriction endonuclease has a four base pair recognition site. In another specific embodiment, the first restriction endonuclease has a recognition site of five, six, seven or eight base pairs. In a further specific embodiment, the first restriction endonuclease is NlaIII, DpnII, Sau3AI, Hsp92II, MboI, NdeII, Bsp1431, Tsp509 I, Hhal, HinP1I, HpaII, MspI, Taqalphal, MaeII or K2091. In a specific embodiment, the second restriction endonuclease has a four base pair recognition site. In another specific embodiment, the second restriction endonuclease has a recognition site of five, six, seven or eight base pairs. In a further specific embodiment, the restriction endonuclease is NlaIII, DpnII, Sau3AI, Hsp92II, MboI, NdeII, Bsp1431, Tsp509 I, HhaI, HinP1I, HpaII, MspI, TaqalphaI, MaeII or K2091. In a specific embodiment, a label is incorporated into the amplified DNA. In a further specific embodiment, the label is incorporated by means of a labeled primer.

In a specific embodiment of the present method in use, the method is performed on DNA derived from a normal cell or tissue and on DNA derived from a different cell or tissue. In a specific embodiment, the method is performed on DNA derived from a normal cell or tissue and on DNA derived from a cancerous cell or tissue. In another specific embodiment, the method is performed on DNA derived from a normal cell or tissue and on DNA derived a cell or tissue treated with a pharmaceutical compound. In an additional specific embodiment, the method is performed on DNA derived from a normal cell or tissue and on DNA derived from a cell or tissue treated with a teratogenic compound. In another specific embodiment, the method is performed on DNA derived from a normal cell or tissue and on DNA derived from a cell or tissue treated with a carcinogenic compound. In an additional specific embodiment, the method is performed on DNA derived from a normal cell or tissue and on DNA derived from a cell or tissue treated with a toxic compound. In another specific embodiment, the method is performed on DNA derived from a normal cell or tissue and on DNA derived from a cell or tissue treated with a biological response modifier. In an additional specific embodiment, the method is performed on DNA derived from a normal cell or tissue and on DNA derived from a cell or tissue treated with a hormone, a hormone agonist or a hormone antagonist. In a specific embodiment, the method is performed on DNA derived from a normal cell or tissue and on DNA derived from a cell or tissue treated with a cytokine. In an additional specific embodiment, the method is performed on DNA derived from a normal cell or tissue and on DNA derived from a cell or tissue treated with a growth factor. In an additional specific embodiment, the method is performed on DNA derived from a normal cell or tissue and on the DNA derived from a cell or tissue treated with the ligand of a known biological receptor. In an additional specific embodiment, the method is performed on DNA derived from a cell or tissue type obtained from a different species. In another specific embodiment, the method is performed on DNA derived from a cell or tissue type obtained from a different organism. In an additional specific embodiment, the method is performed on DNA derived from a cell or tissue at different stages of development. In an additional specific embodiment, the method is performed on DNA derived from a normal cell or tissue and on the DNA derived from a cell or tissue that is diseased. In a further specific embodiment, the method is performed on DNA derived from a cell or tissue cultured in vitro under different conditions. In another specific embodiment, the method is performed on the DNA derived from a cell or tissue from two organisms of the same species with a known genetic difference.

C. Generation of Disease-Specific and Patient-Specific Tumor Models for Personalized Research and Drug Discovery Mesenchymal Chondrosarcoma Cell Lines in Cultures and Xenographs.

MC secondary cell cultures, MC -1 and DM63, were derived from two mouse xenografts (F1), which originated from MC's metastatic lesions. As detailed below, primary MC cell cultures were derived from primary tumors of the lower extremities of an infant (SDMC) and one from a child (LMC).

The initial xenografts were initiated within two hours of surgical resection of mesenchymal chondrosarcoma tissue (F1c) and the in vivo tumors were established over a period of 3-6 months. Subsequently, the in vivo tumors were removed and used to prepare in vitro cultures of the MC cells. In the initial passages, the cells exhibited patterns of biphasic growth consistent with the histopathologic pattern of primary MC, and expression of human HLA antigens was detected by immunofluoresence . Pellets of the cultures stained with H&E showed evidence of a pre-cartilagenous matrix production , and histochemical study showed that the malignant cells produced abundant desmin both in the original tumors and the derived cell cultures . Desmin is a fibrillar protein marker not found in common bone or soft tissue sarcomas. It is normally found only in smooth and skeletal muscles. Its expression in the MC cultures was an indication that the neoplastic cells might be multipotent and originate from a cell of early stem cell origin . RNA expression studies of a complementary xenograft sample showed that receptors for beta FGF were encoded in the malignant cell genome; and growth of the MC -1 was enhanced by use of a mesenchymal cell growth medium with beta FGF supplementation. MC -1 line survived a period of crisis and after 5 months and 7 passages passages entered a phase of accelerated growth. At that time, slender sarcomatous cells predominated and formed “woven” patterns.

DM63 Cell Culture.

A second set of MC xenograft cell cultures was established. Based upon experience gained with MC -1, the primary culture conditions were modified to include matrigel as a depot source of beta FGF and other growth factors. The DM63 cultures entered an accelerated growth phase within 3 passages and were rapidly expanded for cytotoxicity assays. Similar to prior observations with the MC -1, the DM63 also showed regions of biphasic growth

High power microscopy demonstrated an abundance of fine dense cytoplasmic granules which had been less prominent but visible in MC -1. As further evidence of developmental multipotency, adipocyte differentiation was evident in many areas of the primary cultures. This suggested that the cultures were comprised of neoplastic stem cells which could follow a maturation pathway consistent with the sarcoma stem cell model. These accomplishments are of special significance since they suggest that we should be able to purify large numbers of tumor stem cells. These MC tumor stem cells should then be very useful for the discovery of drugs that target the tumor generating cells. Many of the neoplastic stem cells underwent large cell transformation with enormous cytoplasmic lacunae. These lacunae may be sites of mucopolysaccharide accumulation consistent with frustrated cartilage maturation.

SDMC Cell Culture.

In the third set of primary cultures spindle cells grew out rapidly from the explants and >10 passages were achieved within two months of isolation. There was minimal evidence of the biphasic pattern observed in xenograft outgrowths from the adult donor; however the cells again stained strongly positive for desmin .

LMC Cell Culture.

These primary cultures,, showed similar growth, microscopic and immunocytochemical characteristics to the SMDC. One novel feature was the development of three dimensional “baskets” which formed in floating fragments of matrigel and eventually anchored to the plastic substrate. Similar 3D structures subsequently were found to develop when MC -1 or DM63 cells were plated into matrigel. Using a cell migration assay as a surrogate test, both SDMC and LMC exhibited a vigorous invasive potential, indicative of their tumor origins, and were judged to be useful models for chemotherapy testing.

D. Drug and Hormone Testing on Mesenchymal Chondrosarcoma Cells

One objective of the live cell genetics is to test fresh tumor cells for the presence of lineage specific gene expression (hormone or growth factor receptors, specific kinases, etc.) and to relate that information to the response of the tumor cells to various therapies (chemotherapy, antibodies, etc.) and the other diagnostics analyses. Drug and hormone response testing was performed on three kinds of mesenchymal chondrosarcoma fresh tumor cell isolates: primary cultures, low-passage cell lines, and xenograft primary cultures. We were able to start up xenograft and primary cultures of potential value for individualizing MC tumors for chemotherapy options. We preserved, expanded and characterized live MC tumor cells from xenografts through the generation of primary cell lines. While we were trying to grow and expand MC cells, fresh tumor tissue(s) from other MC patients were obtained, in order to characterize their growth requirements, response to chemotherapy drugs, potential to differentiate, and developmental heterogeneity (mesenchymal cells, chondroid cells, desmin positivity etc). With minimal extant information regarding the character of these tumors, we obtained several novel cell cultures. We succeeded in establishing and propagating neoplastic mesenchymal stem cells which appeared to be multipotential with a capacity to differentiate toward one or more sarcoma lineages. This conclusion was based upon several characteristics including not only a pathologist's examination of morphological criteria, which indicated a striking resemblance between the in vitro and in vivo MC tumor growth pattern, but also the production of desmin and other specific tumor antigens in all of the MC tumors, xenografts and primary MC cultures. Desmin is an uncommon tumor marker consistent with inappropriate mesenchymal differentiation towards the smooth muscle lineage. In addition, microscopic examination of early culture passages also indicated production of connective tissue mucopolysaccharide, which is consistent with pre-cartilagenous matrix production. With variable time in culture, the cells grew to sufficient numbers and accelerated in doubling time sufficient enough to support repeated preclinical drug testing; It was apparent, however, that the phase of accelerated growth began more rapidly with samples of childhood tumors or F2 xenografts than with F1 xenografts. Refinement of techniques, including use of matrigel, viability hormones suggested by our Rage genomic studies, and other nutrient substrates accelerated this process.

E. Defining New Diagnostic and Prognostic Biomarkers and Novel Gene or Protein Targets that will Foster Discovery of New Therapeutic Options for these Patients.

A process for Vonfirming the Existence of Mesenchymal Chrondrosarcoma by the over Expression of Fibroblast Growth Factor Receptor Like 1 Gene

Through the studies described in Sections A-D above, the applicant has developed a process for detecting the existence of Mesenchymal Chrondrosarcoma comprising the steps of analyzing tumor cells and determining the extent to which such tumor cells contain fibroblast growth factor receptor-like I protein. When such protein is expressed at least 100-1,000 percent more than in non-cancerous cells, such overexpression is an indicium of the existence of the Mesenchymal Chrondrosarcoma cancer.

In addition, applicant has analyzed the data presented in the Varambally et al. article and discovered that the FGFRL1 protein was aberrantly expressed only with metatstatic prostate cancer and not with the non-metastatic variety or with benign prostate. In any event, one may use the immunoblotting technique described in this article to determine the relative concentrations of the FGFRL1 protein in applicant's process, where one is attempting to determine the existence of Menenchymal Chrondroscarcoma.

I claim:

1. A process for detecting Mesenchymal Chondrosarcoma in a biological organism, comprising detecting, in a sample that contains Mesenchymal Chondrosarcoma cells obtained from a subject a first product indicative of elevated expression of a fibroblast growth factor receptor gene or a second product indicative of elevated amounts of a fibroblast growth factor receptor (FGFR-L1), wherein detection of said first or second product in elevated expression or amount, respectively, compared to a control sample containing normal or benign Mesenchymal Chondrosarcoma cells indicates the presence of Mesenchymal Chondrosarcoma in said subject; and wherein said sample is obtained by a process comprising the steps of:
(a) obtaining a tissue sample from a living biological organism,
(b) disaggregating said tissue sample to produce disaggregated fragments of tissue sample whose maximum dimension is less than about 5 millimeters, wherein said tissue sample is disaggregated within about 10 minutes of the time said tissue sample is obtained from said biological organism, and
(c) disposing said disaggregated tissue fragments in a sterile environment within a container, wherein said sterile environment is comprised of oxygen and a solution comprised of at least one cell type specific viability factor.

2. The process as recited in claim 1, wherein such detection is effected by contacting the sample obtained from the subject with an agent that binds to the extracellular domain of an FGFR L1or with a nucleic acid probe that includes a sequence of at least about 20 nucleotides that hybridizes under conditions of high stringency to nucleic acid encoding the extracellular domain of an FGFR L1.

3. The process as recited in claim 2, where such detection is accomplished by a process comprising the steps of: (a) contacting the sample obtained from said subject with an agent; and (b) detecting the binding of said agent to said product, wherein the detection of the binding of said agent indicates the presence of Mesenchymal Chondrosarcoma.

4. The process as recited in claim 3, wherein said agent is an antibody or a functional fragment thereof.

5. The process as recited in claim 3, wherein said agent is a nucleic acid probe.

6. The process as recited in claim 1, wherein said detection us effected by an immunological process.

7. The process as recited in claim 1, wherein the sample is biopsied tissue.

8. The process as recited in claim 6, wherein an immunological process is used to detect receptor protein.

9. The process as recited in claim 7, wherein said immunological process comprises fixing a sample in paraffin and treating the paraffin-fixed material with an antibody having specific reactivity with the receptor protein, removing unbound antibody from the material, and detecting the antibody bound to receptor protein present in the section.

10. A method for screening biological agents which affect proliferation, differentiation, survival, phenotype, or function of Mesenchymal cells, comprising the steps of:
(a) creating a continuous, adherent, primary cell line derived directly from a Mesenchymal Chondrosarcoma tumor from a first patient with Mesenchymal Chondrosarcoma,
( b) preparing an adherent cell culture of an undifferentiated and differentiated mesenchymal cell population and comprising multipotent mesenchymal tumor stem cells, wherein a single multipotent neural stem cell is capable of producing progeny that are capable of differentiating into cartilaginous cells
(c) contacting said mesenchymal stem cell populations with at least one biological agent or anti-cancer agent, and
(d) determining if said biological or anti-cancer agent has an effect on proliferation, differentiation, survival, phenotype, or function of said Mesenchymal Chondrosarcoma cell population.

11. The method as recited in claim 10, wherein said continuous, adherent, primary cell line is derived directly from a Mesenchymal Chondrosarcoma tumor from a second patient with Mesenchymal Chondrosarcoma.

12. The method as recited in claim 10, wherein said continuous, adherent, primary cell line is derived directly from a Xenograft tumor, and wherein said Xenograft tumor is derived directly from Xenograft tumor that was derived directly from a Mesenchymal Chondrosarcoma tumor from a third patient with Mesenchymal Chondrosarcoma.

13. The method as recited in claim 10, further comprising the step of determining the effects of said biological agent on differentiation of said Mesenchymal Chondrosarcoma stem cell population.

14. The method as recited in claim 10, further compising the step of inducing differentiation of said Mesenchymal Chondrosarcoma stem cell population.

15. The method as recited in claim 10, wherein said mesenchymal stem cell populations are contacted with a biological agent, and wherein said biological agent is a growth factor selected from the group consisting of fibroblast growth factor-1 (FGF-1), FGF-2, epidermal growth factor (EGF), EGF-like ligands, transforming growth factor-.alpha. (TGF.alpha.), insulin-like growth factor (IGF-1), nerve growth factor (NGF), platelet-derived growth factor (PDGF), and TGF.beta.and other growth factors, cytokines or hormones.

16. The method as recited in claim 10, wherein said mesenchymal stem cell populations are contacted with a biological agent, and wherein said biological agent is a trophic factor selected from the group consisting of brain-derived neurotrophic factor (BDNF), ciliary neurotrophic factor (CNTF), and glial-derived neurotrophic factor (GDNF).

17. The method as recited in claim 10, wherein said mesenchymal stem cell populations are contacted with a biological agent, and wherein said biological agent is a regulatory factor selected from the group consisting of phorbol 12-myristate 13-acetate, stauroporine, CGF-41251, tyrphostin, compounds which interfere with activation of the c-fos pathway, compounds which suppress tyrosine kinase activation, and heparan sulfate.

18. The method as recited in claim 10, wherein said mesenchymal stem cell populations are contacted with a biological agent, and wherein said biological agent is a hormone selected from the group consisting of activin and thyrotropin releasing hormone (TRH).

19. The method as recited in claim 10, wherein said mesenchymal stem cell populations are contacted with a biological agent, and wherein said biological agent is a macrophage inflammatory protein (MIP) selected from the group consisting of MIP-1.alpha., MIP-1.beta., and MIP-2.

20. The method as recited in claim 10, wherein said mesenchymal stem cell populations are contacted with a biological agent, and wherein the effect of the biological agent on proliferation of the Mesenchymal Chondrosarcoma cell population is determined by observing changes in size or number of the multipotent neural stem cells.
